(19) United States
(12) Patent Application Publication
(10) Pub. No.: US 2002/0142982 A1
(43) Pub. Date: Oct. 3, 2002

(54) Method for regulating angiogenesis

(76) Inventors: Timothy Hla, Avon, CT (US); Meng-Jer Lee, Unionville, CT (US); Kevin P. Claffey, Burlington, CT (US); Nicolas Ancellin, Farmington, CT (US); Shobha Thangada, Glastonbury, CT (US)

Correspondence Address: CANTOR COLBURN, LLP, 55 GRIFFIN ROAD SOUTH, BLOOMFIELD, CT, 06002

(21) Appl. No.: 09/945,353

(22) Filed: Aug. 31, 2001

Related U.S. Application Data
(63) Continuation-in-part of application No. 09/651,846, filed on Aug. 31, 2000.
(60) Provisional application No. 60/152,266, filed on Sep. 2, 1999.

(51) Int. Cl. A61K 48/00; A61K 31/366

(52) U.S. Cl. 514/44; 514/453

(57) ABSTRACT

Methods for the inhibition of angiogenesis are presented, comprising affecting the response of the EDG-1 receptor by the administration of pharmaceutically effective antagonists of EDG-1 signal transduction. This invention is based in part on the discovery that Akt protein kinase phosphorylation is required for endothelial cell chemotaxis mediated by the EDG-1 G protein-coupled receptor. This invention relates to the use of modifiers of EDG-1 signal transduction to treat disorders of undesired angiogenesis.

### CROSS-REFERENCE TO RELATED APPLICATION

This application is a continuation-in-part of U.S. application Ser. No. 09/651,846, filed on Aug. 31, 2000, which claims priority to U.S. Provisional Application Serial No. 60/152,266, filed on Sep. 2, 1999. The above mentioned applications are herein incorporated by reference in their entirety.

### BACKGROUND OF THE INVENTION

1. Field of the Invention

This invention relates to methods for the inhibition of angiogenesis, and to the use of these methods for the in vivo regulation of angiogenesis, including diagnosis, prevention, and treatment of cancers, disorders and diseases associated with angiogenesis. In particular, this invention relates to compositions and methods for regulating angiogenesis by affecting EDG-1 receptor-mediated signal transduction.

2. Brief Summary of the Background and Related Art

Angiogenesis, the process of new blood vessel formation, is important in embryonic development and many other physiological events, such as wound healing, organ regeneration, and female reproductive processes. During angiogenesis, vascular endothelial cells undergo orderly proliferation, migration, and morphogenesis to form new capillary networks. These events are precisely regulated in vivo by extracellular signals derived from both soluble factors and the extracellular matrix. Because changes in vascularization occur during the menstrual cycle, methods of modifying normal modulation of vascularization are potentially useful in the development of new methods of birth control. The control of angiogenesis is a highly regulated system of angiogenic stimulators and inhibitors. The control of angiogenesis has been found to be altered in certain disease states and, in many cases, the pathological damage associated with the disease is related to the uncontrolled angiogenesis.

Angiogenesis is involved in numerous pathological conditions, such as solid tumor growth, heart disease, rheumatoid arthritis, peripheral vascular diseases of the elderly, diabetic retinopathy, Kaposi's sarcoma, hemangioma, and psoriasis. Angiogenesis is prominent in solid tumor formation and metastasis. Angiogenic factors have been found associated with several solid tumors. A tumor cannot expand without a blood supply to provide nutrients and remove cellular wastes. Prevention of angiogenesis could halt the growth of these tumors and the resultant damage to the animal due to the presence of the tumor. For example, cancerous tumor growth, which depends upon new capillary growth, can be inhibited using compounds that inhibit vascularization, such as angiostatin (O'Reilly, M. S. et al. Cell 79, 315-328 (1994); Folkman, J., Nature Medicine 1: 27-31 (1995)).

Another disease in which angiogenesis is believed to be involved is rheumatoid arthritis. The blood vessels in the synovial lining of the joints undergo angiogenesis. The factors involved in angiogenesis may actively contribute to, and help maintain, the chronically inflamed state of rheumatoid arthritis.

One of the most frequent angiogenic diseases of childhood is the hemangioma. In most cases, the tumors are benign and regress without intervention. In more severe cases, the tumors progress to large cavernous and infiltrative forms and create clinical complications. Systemic forms of hemangiomas, the hemangiomatoses, have a high mortality rate. Therapy-resistant hemangiomas exist that cannot be treated with therapeutics currently in use.

In addition, since endothelial cell injury can lead to heart attacks, stimulation of growth and repair of endothelial cells and the structures they comprise are important to keep the cardiovascular system healthy. For example, ischemic heart tissue, in which the blood supply is inadequate, can be treated by surgically inducing transmyocardial revascularization. In this procedure, ablation of heart tissue locally stimulates growth of new capillaries. This method involves puncturing the heart wall to form channels (Korkola, S., et al., J. Formos Med. Assoc. 98: 301-308 (1999)). Because current methods of modulating angiogenesis, such as transmyocardial revascularization, involve surgical intervention and cell destruction, there remains a need for methods of inducing and inhibiting angiogenesis that are highly specific for endothelial cells and do not involve tissue ablation. In addition, a method for stimulating growth and repair of endothelial cells may be important to keep the cardiovascular system healthy.

A few methods for the modulation of angiogenesis have been disclosed. U.S. Pat. No. 6,025,331 to Moses et al. discloses a method for treating disorders arising from abnormal angiogenesis comprising administration of troponin subunits C, I, and T, which inhibit endothelial cell proliferation. U.S. Pat. No. 5,851, to Ulrich et al. discloses use of a pharmaceutical composition comprising an expression vector for FLK-1 tyrosine kinase receptor. There nonetheless remains a need in the art for the regulation of angiogenesis in both normal and pathological physiological events.

Cultured endothelial cells such a human umbilical vein endothelial cells (HUVEC) are accepted in vitro model systems for studying angiogenesis. Cultured endothelial cells migrate and proliferate in response to angiogenic growth factors, such as fibroblast growth factor-1 (FGF-1), FGF-2 and vascular endothelial growth factor (VEGF). A basement membrane extract derived from the Engelbreth-Holm-Swarm (EHS) mouse sarcoma (available from Collaborative Research under the trade name MATRIGEL) promotes morphogenesis of endothelial cells into capillary-like structures in the presence of angiogenic factors and serum in vitro. Furthermore, addition of phorbol 12-myristic 13-acetate (PMA) to endothelial cells grown on 3-dimensional collagen or fibrin gels results in the formation of networks of capillary-like structures. PMA treatment of HUVEC is also known to induce expression of a G-protein-coupled receptor (GPCR), coded for by the endothelial differentiation gene-1 (EDG-1). Stable transfectants of EDG-1 in human embryonic kidney 293 (HEK293) cells (HEK293EDG-1) will furthermore differentiate endothelial morphology when an EDG-1 ligand is presented. Several other GPCRs related in primary sequence to EDG-1 have been isolated, including EDG-2/VZG-1, EDG-3, EDG-4, EDG-5/H218/AGR16 and EDG-6. The EDG family of receptors differs in tissue distribution.

Because these receptors are coupled to a G protein, functional assays such as changes in calcium levels and stimulation of intracellular kinases can be used to elucidate the relationship between GPCR-ligand binding and cellular responses. The morphogenetic response of HEK293EDG-1 cells to EDG-1 activation can be used as an assay for screening EDG-1 ligands. Investigations using this assay led to identification of the serum-borne lipid sphingosine-1-phosphate as an EDG-1 agonist by Lee et al., in Science, Vol. 279, 1552-55 (1998). Specific ³²P-SPP binding was observed only in HEK293EDG-1 cells (dissociation constant, K_{d}=8.1 nM) but not in HEK293 control cells. SPP binding to EDG-1 activated mitogen-activated protein (MAP) kinase, and induced EDG-1 receptor phosphorylation and internalization. These data show that EDG-1 is a high affinity, plasma membrane-localized receptor for SPP. Other data have shown that EDG-3 and EDG-5 respond to low concentrations of SPP in a Xenopus oocyte-based calcium efflux assay and serum response factor assay in Jurkat T-cells (An et al., FEBS Lett. Vol. 417, 279-282 (1997)). However, the mode of action of SPP remains an open question, particularly as to whether the various actions of SPP are due to its role as an extracellular mediator that signals via plasma membrane receptors, whether it acts intracellularly as a second messenger molecule, or a combination of the two.

Chemotaxis is a complex, orchestrated phenomenon that is stimulated by extracellular ligands acting on their cell surface receptors, e.g., G protein-coupled receptors (GPCR) and receptor tyrosine kinases (RTK). Polarized localization of signaling molecules such as the protein kinase Akt (also known as protein kinase B) appear to be important for chemotaxis. The molecular mechanisms by which Akt regulate chemotaxis are not clear. The protein kinase Akt phosphorylates substrates with the consensus sequence of (RxRxxS/T). It is of interest that many angiogenic factors, such as vascular endothelial cell growth factor (VEGF) and angiopoietin, utilize the Pl-3-kinase/Akt signaling pathway to regulate endothelial cell behavior important in angiogenesis-for example, cell migration and survival. Endothelial cell chemotaxis is controlled by numerous angiogenic factors, such as VEGF and fibroblast growth factor (FGF), as well as by bioactive lipids such as sphingosine 1-phosphate (S1P). S1P, a product of sphingomyelin metabolism, mediates its actions by interacting with GPCRs of the EDG-1 family. Activation of EDG-1 regulates intracellular signaling pathways, which results in endothelial cell migration. Specific molecular mechanisms regulated by the EDG receptors that are required for cell migration are not defined. Specifically, how EDG receptors regulate rapid cellular changes, for example, calcium transients, ERK phosphorylation, and transition, into long-term changes in cell behavior, such as cell migration and survival, is not understood.

### SUMMARY OF THE INVENTION

In one embodiment, the need for an improved method for modulating angiogenesis is met by administration of a pharmaceutically effective quantity of sphingosine-1-phosphate, sphingosine-1-phosphate analogs, and other agonists of EDG-1, EDG-3, EDG-5, or a combination comprising at least one of the foregoing receptors. Another embodiment of the present invention accordingly comprises a pharmaceutically effective composition comprising sphingosine-1-phosphate, sphingosine-1-phosphate analogs, and other agonists of EDG-1, EDG-3, EDG-5, or a combination comprising at least one of the foregoing receptors. Administration of such compositions is particularly effective to stimulate angiogenesis, endothelial cell survival, and intercellular junction formation.

In another embodiment, a method for the modulation of angiogenesis comprises construction and administration of vectors comprising antisense oligonucleotides effective to inhibit expression of EDG-1, EDG-3, or a combination comprising at least one of the foregoing receptors. Another embodiment of the present invention accordingly comprises a pharmaceutically effective composition comprising antisense oligonucleotides effective to inhibit expression of EDG-1, EDG-3, or a combination comprising at least one of the foregoing receptors.

In another embodiment, a gene therapy method comprises construction and administration of vectors effective to overexpress EDG-1, EDG-3, or a combination comprising at least one of the foregoing receptors in the endothelial cells of the body in an amount effective to induce angiogenesis.

In yet another embodiment, a gene therapy method comprises construction and administration of vectors effective to inhibit expression of EDG-1, EDG-3, or a combination comprising at least one of the foregoing receptors in the endothelial cells of the body in amount effective to inhibit angiogenesis.

In another embodiment, a method to inhibit angiogenesis comprises administration of an effective quantity of an antagonist of EDG-1 signal transduction. Another embodiment is to use a PI-3 kinase inhibitor or an Akt kinase inhibitor as the antagonist of EDG-1 signal transduction.

In another embodiment, the antagonist of EDG-1 signal transduction is used to treat undesired angiogenesis in tumors, rheumatoid arthritis, diabetic retinopathy, Kaposi's sarcoma, hemangioma or psoriasis.

In another embodiment, the antagonist of EDG-1 signal transduction is an anti-EDG-1 antibody. A preferrred anti-EDG-1 antibody is a chicken anti-human EDG-1 antibody or a biologically active fragment thereof.

The invention is further illustrated by the following drawings and detailed descriptions. All references mentioned herein are hereby incorporated by reference in their entirety.

### BRIEF DESCRIPTION OF THE FIGURES

FIG. 1 shows Northern Blots illustrating expression of EDG-1 and EDG-3 in endothelial cells, showing poly(A)⁺ RNA from HUVEC (lane 1) and HEK293 (lane 2) probed with EDG-1, EDG-3, EDG-5, and GAPDH cDNAs. A positive control comprising in vitro transcripts for EDG-1, -3, and -5 (+VE) is shown in lane 3.

FIGS. 2A and 2B shows graphs illustrating SPP-induced intracellular calcium, wherein some cells were pretreated with PTx (500 ng/mL) for 16 hours.

FIG. 3 illustrates that the presence of SPP in endothelial cells induces Gᵢ-dependent MAP kinase activation.

FIG. 4 shows fluorescence microscope images of HUVEC cells treated with C3 exoenzyme (first and second rows) or N17Rac (third row) and then treated with or without SPP. The left column shows visualization with FITC-IgG (left column) and of the actin microfilaments (right column).

FIG. 5 shows fluorescence microscope images of HUVEC cells treated with or without SPP. The left column shows visualization for VE-cadherin and the right column shows visualization for β-cadherin (scale bar=13.4 microns).

FIG. 6 illustrates fractionation of HUVEC cell lysates into Triton-X-100-soluble and -insoluble fractions wherein unstimulated HUVEC (−) or HUVEC stimulated with 500 nM SPP for 1 hour (+) were sequentially fractionated with TX-100 (0.05, 0.1, 0.5%). Equal amounts of protein extracts were loaded and probed with ant-VE-cadherin antibody (upper panel). HUVEC were stimulated with 500 nM of SPP for the indicated times, extracted with 0.5% Triton-X-100, the insoluble fractions were further extracted with 1% Triton-X-100 plus SDS, and probed for VE-cadherin by Western blot (middle panel).

FIG. 7 are SDS-PAGE gels of HUVEC labeled to steady state with ^{35S}methionine (250 μCi/mL, NEN DuPont) for 24 hours, stimulated with 500 nM SPP for 1 hour, fractionated with 0.5% TX-100, centrifuged, and the protein complexes in detergent-insoluble fractions cross-linked with 0.5 mM Dithiobis[succinimidyl propionate], extracted with 1% TX-100 and cell extracts were immunoprecipitated with antibodies to VE cadherin, β-catenin, γ-catenin, or p120 Src. (An unidentified polypeptide of about 80 Kd(*) was also co-immunoprecipitated.

FIGS. 8A-B are fluorescence microscope images of HUVEC after stimulation with 500 nM SPP for 30 minutes, immunostained with antibodies against Rac, Rho, and/or the Rho-specific guanine nucleotide exchange factor Tiam 1. Primary antibody binding was imaged using FITC-conjugated goat anti-rabbit and/or TRITC-conjugated sheep anti-mouse.

FIGS. 9A-C illustrate (A) induction of morphogenesis in cultured endothelial cells; (B) a quantitative analysis of tubular length in response to SPP, Spp+PTx, SPP+C3, SPM, and C1P; and (C) a quantitative analysis of tubular length in response to SPP with VE-cadherin.

FIGS. 10A-B illustrate (A) HUVEC treated with C₂-ceramide in the absence (C₂-Cer) or presence of 500 nM SPP (C₂-Cer+SPP); and (B) HUVEC incubated with ^{3H}methyl-thymidine, SPP+PTx, or SPP+PD98059; then washed before exposure to C₂-Ceramide in the presence or absence of SPP.

FIGS. 11A-B show (A) low power micrographs of MATRIGEL plugs implanted into athymic mice; (B) quantification of neovessels; and (C) transmission electron micrographs of SPP-induced neovessels, wherein a. is vehicle, b. is FGF-2, and c. is FGF-2+SPP, each Figure demonstrating that SPP potentiates FGF-2-induced angiogenesis in vivo.

FIG. 12 shows the sequences of phosphothioate oligonucleotides having sequence identification numbers 1-8.

FIG. 13 is data demonstrating the efficacy and specificity of EDG-1 and EDG-3 PTOs wherein Xenopus oocytes were injected with in vitro transcribed RNA and the indicated PTO, and Ca²⁺ rises induced by SPP were performed as described (Ancellin, N., and Hla, T., J. Biol. Chem. 274: 18997-19002 (1999)); n=number of oocytes injected.

FIG. 14 are fluorescence microscopy images showing that EDG-1 and EDG-3 expression is required for SPP-induced adherens junction assembly, wherein HUVEC were microinjected with antisense (αs) or sense(s) PTO (20 μM in the micropipette) for EDG-1 and EDG-3, and 18 to 24 hours thereafter, cells were stimulated with 0.5 μM SPP for 1 hour, fixed, and VE-cadherin localization determined; FITC-IgG column indicates the microinjected cells, and VE-cad panels indicate the signal for VE-cadherin in the same microscopic field; scale bar represents 16 microns.

FIG. 15 are fluorescence microscopy images illustrating EDG-1 and EDG-3 regulation of SPP-induced cytoskeletal dynamics, wherein HUVEC were microinjected with EDG-1 and -3 PTO, and the actin cytoskeleton was labeled with TRITC-phalloidin. Microinjected cells are marked with the FITC-IgG (left column). The EDG-1 antisense PTO specifically inhibited cortical actin (arrows indicate injected cells, arrowheads, uninjected cells) whereas the EDG-3 PTO blocked stress fiber formation (asterisks indicate injected cells). Scale bar indicates 17 microns.

FIG. 16 shows graphs illustrating that EDG-1 and EDG-3 PTOs inhibit SPP-induced morphogenesis, wherein individual PTOs (0.2 μM in upper panel) was delivered into HUVEC by Lipofectin reagent, and after 24 hours, cells w ere trypsinized, plated onto MATRIGEL in the absence or presence of SPP (500 nM) and tubular length was quantitated.

FIG. 17 is a graph showing the effect of EDG-1 and EDG-3 PTOs and VEGF on SPP-induced angiogenesis; αSEDG, antisense EDG-1 (19.2 μM)+antisense EDG-3 (4.8 μM); SEDG, sense EDG-1 (19.2 μM)+sense EDG-3 (4.8 μM); FGF, 1.3 μg/mL; SPP, 500 nM; VEGF, 1.4 μg/mL; (*),FGF+SPP+antisense vs. FGF+SPP (p<0.05, test); (**), VEGF, +SPP vs. VEGF (p<0.05, t test).

FIG. 18 is an immunoblot performed with anti-AKT antibody that shows GST fused to EDG-1-i₃ but not GST alone interacts with Akt.

FIG. 19 is an in vitro phosphorylation assay that demonstrates that EDG-1-i₃ is specifically phosphorylated by Akt.

FIGS. 20A and B show the identification of the EDG-1-i₃ residue that is phosphorylated by Akt. Trypsin digestion of radioactively labeled EDG-1-i₃ followed by chromatography on a C18 column (A) reveals one major labeled tryptic phosphopeptide. Phosphoamino acid analysis (B) reveals that the tryptic peptide contains only phosphothreonine.

FIG. 21 shows the results of solid phase sequencing of the tryptic peptide. The amino acid sequence is identified as residues 234-238 with the phosphorylation site at T²³⁶.

FIG. 22 is an SDS PAGE showing that mutation of T236 to V236 significantly reduces the incorporation of radioactive phosphate into EDG-1-i₃.

FIG. 23 is an immunoprecipitation experiment showing that S1P or RTK influences the association between EDG-1 and Akt. HEK293pCDNA (cont.) and HEK293EDG-1 (EDG-1) cells were stimulated without or with S1P (100 nM) and IGF-1 (50 ng/ml) for 1 hr. Extracts were immunoprecipitated with anti-M2 to pull down EDG-1, followed by immunoblotting with anti-phospho-Akt (first panel). The blot was reprobed with anti-M2 to show the precipitated EDG-1 (second panel). The level of phospho- and total Akt in extracts was determined by immunoblotting (third and fourth panels).

FIG. 24 is an immunoprecipitation experiment showing that EDG-1 but not EDG-3 or EDG-5 associates with Akt and that the EDG-1-Akt association is enhanced when Akt is activated by S1P and IGF-1. HEK293 cells were co-transfected with pCDNA or Flag-tagged EDG receptors (EDG-1, or -3 or -5), along with HA-tagged wild-type Akt (WT-Akt). After stimulation, extracts were immunoprecipitated with anti-M2 followed by immunoblotting with anti-HA (upper panel). The blot was reprobed with anti-M2 (lower panel). Equal expression of Akt polypeptides was verified by immunoblotting extracts with anti-HA (data not shown).

FIG. 25 is an immunoprecipitation experiment showing that dominant-negative Akt does not associate with EDG-1 while constitutively active EDG-1 binds Akt even in the absence of S1P and IGF-1. HEK293 cells were co-transfected with Flag-tagged EDG-1 and HA-tagged wild-type, dominant-negative (DN), or constitutively-active (Myr) Akt plasmids (Alessi et al., 1996). After stimulation, the presence of Akt in EDG-1 immunoprecipitates was examined by an anti-HA immunoblot (first panel). Second panel, the precipitated EDG-1; third panel, equal expression of Akt in transfectants. −Ve=extracts from untransfected HEK293. Also, extracts were immunoprecipitated with anti-HA followed by in vitro kinase assay using H2B as substrate. The fourth panel shows the autoradiogram of phosphorylated H2B.

FIG. 26 is an SDS PAGE showing that S1P treatment induces EDG-1 phosphorylation. HEK293EDG-1 were labeled with [³²P]-orthophosphate, and stimulated with 100 nM S1P for indicated times. Some cultures were treated with 10 μM LY294002.

FIG. 27 is an immunoprecipitation experiment that demonstrates that Akt also phosphorylates endogenous EDG-1. (Upper panel) Normal HUVEC cells were labelled with [³²P]-orthophosphate, and stimulated with 100 nM S1P for indicated times. Cell extracts were immunoprecipitated with the affinity-purifed anti-EDG-1 IgY, separated on a SDS-PAGE and autoradiographed. (Middle panel) Some cells were treated with 10 μM LY294002 or 100 nM Wortmannin prior to stimulation with S1P and EDG-1 phosphorylation was examined as above. (Lower panel) Some cells were transduced with 100 MOI of wild-type (wt)-Akt, dominant negative (dn)-Akt or the β-gal virus for 12 h prior to stimulation with S1P. EDG-1 phosphorylation was examined as above.

FIG. 28 is an immunoblot assay showing that endogenous EDG-1 and Akt associate. HUVEC cells were stimulated with S1P (100 nM), VEGF (10 ng/ml) and IGF-1 (50 ng/ml) for 30 min. Cell extracts were immunoprecipitated with the affinity-purified anti-EDG-1 IgY, and analyzed by an immunoblot assay for endogenous Akt or EDG-1 polypeptides.

FIG. 29 are fluorescence microscopy images showing that Akt activation by S1P is important for cortical actin assembly in HUVAC. HUVEC were transduced with adenoviral vectors carrying β-gal (cont.), wild-type (Akt^{WT}), dominant negative (Akt^{DN}), or constitutively-active (Akt^{Myr}) Akt. After treatment without or with 100 nM SPP, actin cytoskeleton was visualized by TRITC-Phalloidin staining. Note that S1P induced both cortical actin (arrow) and stress fibers (arrowhead) in β-gal and Akt^{WT} infected HUVEC. However, S1P only induced stress fibers in Akt^{DN} infected cells (arrowhead in third row). Also note that Akt^{Myr} induced cortical actin (arrow), but not stress fibers, in the absence of S1P. Treatment of S1P significantly induced stress fibers formation in Akt^{Myr} infected cells (arrowhead). Scale bar 19 μM.

FIG. 30 is an immunoblot analysis showing the specificity of dominant negative Akt overexpression. HUVEC were transduced with either the β-gal virus (cont.) or dominant negative Akt (Akt^{DN}) virus and stimulated with S1P. Extracts were analyzed by immunoblot analysis with indicated phospho-specific antibodies.

FIG. 31 shows the role of Akt in EDG-1-induced cortical actin assembly and cell migration. Upper panel; CHO cells stably transfected with pCDNA (C), EDG-1 (El), EDG-3 (E3), EDG-5 (E5), were treated or not with 100 nM S1P, and cell migration was quantified as described. Middle panel, CHO transfectants were transduced with adenovirus vectors encoding β-Gal or dominant negative Akt for 16 h, cell migration was then measured in the absence or presence of S1P. Anti-HA immunoblot on cell extracts shows the equal expression of dominant negative Akt polypeptides. Lower panel, Migration assays on EDG-1-expressing CHO cells were performed in the presence of S1P and various inhibitors: Wort, Wortmannin (100 nM); LY, LY294002 (10 μM); Rapa, Rapamycin (100 nM).

FIG. 32 is a graph showing the Ca²⁺ rise in Xenopus oocytes expressing EDG-1 and the heterotrimeric Gqi protein. Xenopus oocytes were injected with in vitro transcribed RNA of EDG-1 or the Akt phosphorylation site mutant (T236A) EDG-1^{TA} and the G_{qi} protein, and Ca⁺² rises induced by S1P (90 sec) were quantitated.

FIG. 33 is an immunoblot showing stimulation of G-dependent protein kinases. CHO cells were stably-transfected with pCDNA, wild-type EDG-1 (EDG-1^{WT}), or mutant EDG-1 (EDG-1^{TA}). After stimulation with 100 nM S1P, extracts were immunoblotted with indicated phospho-specific antibodies. Western-blotting with B-actin antibody shows equal amount of extracts loaded.

FIG. 34 is an immunoblot showing EDG-1 mutant receptor association with Akt. HEK293 were co-transfected with EDG-1 or T236A EDG-1, together with wild-type Akt. After stimulation with S1P (100 nM, 30 min), the Akt association was examined.

FIG. 35 is an SDS-PAGE showing phosphorylation of mutant EDG-1 receptors. HEK293 cells were transfected with flag-tagged wild-type EDG-1 or T236A, R233K, R231K mutants. Expressed receptors were immunoprecipitated with anti-M2 and phosphorylated in vitro with (1 U/ml) active Akt or p90^{RSK} and [³²P]-□-ATP. Phosphorylated proteins were analyzed by SDS-PAGE and autoradiography. Expression of the receptors was assayed by immunoblotting cell extracts with anti-M2 antibody. The activity of the kinases was measured by phosphorylation of histone 2B (H2B).

FIGS. 36A and B are graphs showing the role of the mutant EDG-1 receptors in EDG-1 signaling. S1P-induced chemotaxis in CHO cells stably transfected with EDG-1^{WT}, and Akt phosphorylation mutants (R231K and R233K) (A). CHO cells stably transfected with pCDNA, EDG-1^{WT} or EDG-1^{TA} were stimulated with various doses of S1P and chemotaxis was quantified (B).

FIG. 37 are fluorescence microscopy images of CHO cell lines expressing the mutant EDG-1 receptors. TRITC-Phalloidin staining showed that the reorganization of actin cytoskeleton in CHO cells stably expressing wild-type, T236A EDG-1, and EDG-3 receptors. Note that S1P-induced cortical actin formation was observed only in EDG-1 cells (arrows in second row). Also note that S1P is unable to activate the formation of cortical actin in T236A EDG-1 cells. Scale bar=26 μM.

FIGS. 38A and B show the effect of the mutant EDG-1 receptor on Rac GTPase activation. Defective Rac activation by the T236AEDG-1 mutant. S1P-induced Rac activation in CHO cells expressing wild-type EDG-1 (WT) or the T236A mutant (TA) was measured as described. Total Rac was measured in cell extracts by immunoblot analysis. (A) The effect of prior transduction of adenoviral particles encoding the T236AEDG-1 (EDG-1^{TA}), wild-type (WT)-Akt, dominant negative (DN)-Akt and the β-gal on Rac activation in CHO-EDG-1 cells is quantified and plotted (B).

FIGS. 39A and B are graphs of cell migration in cell lines expressing EDG-1 (A) or EDG-3 (B). CHO cells stably expressing EDG-1 (A) and EDG-3 (B) were transduced with indicated MOI of β-gal, wild-type, or T236A EDG-1 adenoviral particles. Cell migration responses to S1P were then quantified. The inset in (A) shows the anti-M2 immunoblot of CHO cells transduced with β-gal (cont.), wild-type (WT), or T236A (TA) EDG-1 viruses.

FIG. 40 is a graph showing migration of HUVEC cells transduced with the mutant EDG-1 receptor under a variety of conditions. HUVEC cells were transduced with β-gal or T236AEDG-1 viruses. S1P-induced migration was then measured. C=untreated HUVEC, S1P=stimulated with 100 nM S1P, TA=transduced with T236AEDG-1 virus, □-gal=transduced with β-gal virus.

FIG. 41 is a graph showing the effect of increasing Akt levels to overcome suppressive effect of the EDG-1 mutant. S1P-induced migration was conducted in HUVEC transduced with T236AEDG-1 virus (10 MOI) and increasing MOI of wild-type Akt virus.

FIG. 42 is microscope images showing morphogenesis of HUVEC cells plated on Matrigel. HUVEC cells were transduced with 50 MOI of indicated viruses; a,b=none, c=β-gal, d=wild-type EDG-1, e=T236AEDG-1, f=T236AEDG-1+wild-type Akt. Total viral load in c-f is normalized. In vitro angiogenesis assay on Matrigel was conducted in the presence (b to f) or not (a) of 100 nM S1P. The numbers are mean±s.e.m of total tubular length per microscopic field (n=2). Scale bar=170 μm.

FIG. 43 is a histological analysis of matrigel model of angiogenesis in nude mice. Matrigel plugs supplemented with S1P (500 nM), VEGF (0.7 μg/ml) and FGF-2 (1.4 μg/ml) were mixed with 2×10⁹ pfu/ml of wild-type (WT) or the T236A (TA) EDG-1 virus and injected subcutaneously into Nude mice. The angiogenic response was assessed by histological sectioning of the Matrigel plugs. Representative photomicrographs show the invasive vascular front in the Matrigel plug was inhibited by the T236AEDG-1 virus transduction. SM=skeletal muscle in the subcutaneous area, MG=Matrigel plug. Arrows denote mature vasculature. Scale bar=50 μm. The microvessel counts are: vehicle=0.92±0.42; S1P/VEGF/FGF plus β-gal virus=20.48±3.93; S1P/VEGF/FGF plus EDG-1 wild-type virus=21.38±2.9; S1P/VEGF/FGF plus T236AEDG-1 virus=13.0±4.8. S1P/VEGF/FGF plus EDG-1 wild-type virus versus S1P/VEGF/FGF plus T236AEDG-1 virus is significantly different (p<0.02, Student's t-test; n=3).

### DETAILED DESCRIPTION OF THE PREFERRED EMBODIMENTS

The present invention is based in part on the discovery that sphingosine-1-phosphate (SPP) and sphingosine-1-phosphate analogs are extracellular modulators of angiogenesis through the G-protein coupled receptors EDG-1, EDG-3, and EDG-5.

As stated above, it has been found that in addition to EDG-1, EDG-3 and EDG-5 are high affinity receptors for SPP. (EDG-2 and EDG-4 appear to be lysophosphatidic acid (LPA) receptors.) EDG-1 couples to Gᵢ but is unable to couple to the heterotrimeric G_{q} protein, whereas EDG-3 potently activates G_{q}. EDG-5 appears to couple to the G_{q} pathway, albeit less effectively that EDG-3. Both EDG-3 and EDG-5, however, are also capable of coupling to the Gᵢ pathway. Thus, it appears that EDG-1, -3 and -5 are subtypes of SPP receptors which couple to different signaling pathway and thus likely regulate different biological responses.

SPP binding to the EDG-1, EDG-3, and EDG-5 receptors not only activates the receptors, it also transduces intracellular signal transduction and thus regulates specific biological responses. EDG-1 is highly expressed in vascular endothelial cells in vitro and its expression is correlated with endothelial cell differentiation in vitro. These observations suggest that SPP interaction with the EDG-1, EDG-3, and EDG-5 receptors play an important role in normal development and wound healing.

In particular, bioactive lipids such as SPP and LPA regulate cytoskeletal architecture by signaling through the Rho family of GTPases. It has been discovered that in endothelial cells SPP acts as an extracellular mediator to induce actin stress fibers and cortical actin. Induction of stress fibers requires the activity of Rho whereas dominant negative Rac inhibited both stress fibers and cortical actin assembly. SPP effects on the cytoskeleton are not inhibited by pertussis toxin. These data suggest that SPP interaction with HUVEC regulates Rho and Rac activity by a Gᵢ-independent pathway.

Significantly, SPP treatment of HUVEC regulates the translocation of Tiam 1 (an upstream activator of Rac) and Rac to cell-cell junctions. Furthermore, VE-cadherin and catenin molecules are also translocated to discontinuous structures at cell-cell junctions in response to SPP. Moreover, VE-cadherin partitions into a detergent insoluble fraction after SPP treatment, suggesting that SPP induces adherens junction assembly in HUVEC. Indeed, immunoprecipitation experiments suggest that detergent insoluble β- and -catenin are found associated with other adherens junction proteins and VE-cadherin after SPP treatment. These data indicate that the adherens junctions in endothelial cells are under dynamic control by SPP signaling as an extracellular mediator. In contrast, polypeptide cytokines such as VEGF and TNF-α are known to disrupt adherens junctions, a phenomenon which may be responsible for enhanced vascular permeability and increased extravasation of blood-borne cells. Therefore, under physiological conditions, SPP may promote endothelial cell integrity and functionality.

SPP-stimulated translocation of VE-cadherin and γ-catenin to cell-cell junctions requires the activity of Rho and Rac GTPases. Similar to the regulation of actin cytoskeleton, microinjection of SPP into HUVEC cells did not regulate VE-cadherin and β-catenin translocation, suggesting that extracellular action of SPP on plasma membrane receptors is involved. In addition, pertussis toxin treatment did not inhibit VE-cadherin and β-catenin translocation, suggesting that a non-Gᵢ pathway is involved. These data agree with previous findings in epithelial cells and keratinocytes that adherens junction assembly requires the activity of Rho and Rac. However, a recent report showed that Rho and Rac are not required to maintain confluence-induced adherens junctions in endothelial cells. These data suggest that multiple mechanisms are involved in adherens junction formation and maintenance. Rho is thought to control stress fibers and cytoskeletal contraction whereas Rac appears to control cortical actin assembly. That Tiam 1 and Rac co-localizes with β-catenin after SPP treatment suggest that it may directly participate in the linkage of cadherin complexes to the cytoskeleton. Mechanistic details of how GPCRs regulate Rho and Rae activity are not well understood. The G₁₃ family of heterotrimeric G-proteins has been implicated in Rho activation, stress fiber and focal adhesion assembly. Although some GPCRs may activate Rho via G₁₃, a recent study has shown that certain GPCRs may directly bind and activate Rho via the NpxxY motif. Because EDG-1 is the major SPP receptor in HUVEC, a non-Gi coupling activity of EDG-1 may regulate Rho and Rac activity. However, the contribution of low-level expression of EDG-3 cannot be completely ruled out. Alternatively, cooperative signaling of EDG-1 and low levels of EDG-3 may be important. Nevertheless, the data indicate that plasma membrane receptors and not intracellular receptors for SPP are critical for endothelial cell responses.

In addition, SPP protects endothelial cells potently from apoptosis induced by ceramide, 15d-PGJ₂ and growth factor withdrawal. These treatments are known to induce caspase-dependent apoptosis. SPP was previously shown to protect monocytic cells from ceramide-induced apoptosis, which was interpreted to occur via a second messenger action. In this study, we show that nanomolar concentrations of extracellular SPP prevented endothelial cell apoptosis. This effect was completely blocked by pertussis toxin and the MAP kinase inhibitor PD98059, suggesting that SPP signaling via the Gᵢ pathway is involved. These data also suggest that SPP may be an important serum-borne survival factor for endothelial cells, given that the K_{d} of SPP of EDG-1 is 8 nM and plasma concentrations were estimated to be 100 nM.

SPP induced endothelial cell morphogenesis into capillary-like networks in the MATRIGEL model of in vitro angiogenesis. This was potently inhibited by VE-cadherin extracellular domain antibodies, pertussis toxin and C3 exotoxin. These data strongly suggest that the adherens junction assembly and the protection of endothelial cell morphogenesis is a complex process which requires the interaction of cells with the extracellular matrix, directed migration, cell-cell interactions, and perivascular proteolysis, among others. Indeed, inhibition of critical cell-matrix interaction molecules such as αᵥβ₃ with blocking antibodies result in endothelial cell apoptosis and vessel regression. Other studies have also implicated the importance of VE-cadherin in endothelial cell morphogenesis. The present data suggest that adherens junction assembly is under dynamic control by SPP signaling via its GPCRs and is required for endothelial cell morphogenesis and survival.

In vivo data further support these conclusions. Implantation of SPP- and FGF-2-containing MATRIGEL plugs into athymic mice resulted in significant potentiation of s-2-induced angiogenesis. Neovessels formed in the presence of SPP are mature and contain well-developed adherens junctions. However, SPP alone did not induce significant angiogenesis. Mechanistically, SPP likely acts distinctly from VEGF, a potent and specific inducer of endothelial cell permeability and migration. Vessels formed in the presence of VEGF are often leaky and are not functional optimally. The present data suggest that SPP is a modulator of angiogenesis which acts at later phases, that of cell-cell junction assembly, morphogenesis and inhibition of apoptosis. Endogenous production of SPP by thrombotic platelets and signaling via the EDG-1 pathway may be an important aspect of the angiogenesis process.

Based on these data, one embodiment of the present invention is an improved method for regulating angiogenesis comprising administration of a pharmaceutically effective quantity of SPP or its pharmaceutically acceptable salts or esters, SPP analogs or their pharmaceutically acceptable salts or esters, or a combination thereof. Analogs of SPP include the corresponding acids, salts, and esters of dihydrosphingosine 1-phosphate; analogs wherein phosphonate, phosphinate, carboxylate, sulfonate, sulfinate, or other negatively-charged ionic groups are substituted for the phosphate group; methylated derivatives such as phosphorylated cis-4methylsphingosine; and sphingosyl phosphoryl choline.

As administration of SPP or SPP analogs which activate EDG-1 and EDG-3 receptors induce angiogenesis, such administration is effective to accelerate wound healing in diabetic ulcers, stomach, and other gastrointestinal ulcers. It may also be effective to induce new vessel growth in the myocardium of the heart suffering from reduced blood supply due to ischemic heart disease, thereby providing a useful alternative to ablative surgery.

It has also been shown that presence of SPP induces the formation of stress fibers and cortical actin through regulation of the activity of Rho and Rac small GTPases, respectively. Administration of SPP and SPP analogs may therefore further be used to induce endothelial cell survival and intercellular junction formation, thereby repairing endothelial cell injury or preventing toxicity.

Methods for the formulation of pharmaceutically acceptable compositions comprising SPP, its salts and derivatives, and SPP analogs, and its salt and derivatives are generally known. The subject pharmaceutical formulations may comprise one or more non-biologically active compounds, i.e., excipients, such as stabilizers (to promote long term storage), emulsifiers, binding agents, thickening agents, salts, preservatives, and the like, depending on the route of administration.

For oral administration, SPP, its salts and derivatives, and SPP analogs, their salts and derivatives may be administered with an inert diluent or with an assimilable edible carrier, or incorporated directly with the food of the diet. The formulations may be incorporated with excipients and used in the form of ingestible tablets, buccal tablets, troches, capsules, elixirs, suspension syrups, wafers, and the like. The tablets, troches, pills, capsules and the like may also contain the following: a binder, such as gum tragacanth, acacia, cornstarch, or gelatin; excipients, such as dicalcium phosphate; a disintegrating agent such as corn starch, potato starch, alginic acid and the like; a lubricant such as magnesium stearate; and a sweetening agents, such as sucrose, lactose or saccharin; a flavoring agent such as peppermint, oil of wintergreen, or the like flavoring. When the dosage unit is a capsule, it may contain, in addition to materials of the above type, a liquid carrier. Various other materials may also be present as coatings or to otherwise modify the physical form of the dosage unit. A syrup or elixir may contain sucrose as a sweetening agent, methyl and propylparabens as preservatives, a dye and flavoring such as cherry or range flavor. Such additional materials should be substantially non-toxic in the amounts employed. Furthermore, the active agents may be incorporated into sustained-release preparations and formulations. Formulations for parenteral administration may include sterile aqueous solutions or dispersions, and sterile powders for the extemporaneous preparation of sterile, injectable solutions or dispersions. The solutions or dispersions may also contain buffers, diluents, and other suitable additives, and may be designed to promote the cellular uptake of the active agents in the composition, e.g., liposomes. Sterile injectable solutions are prepared by incorporating the active compounds in the required amount in the appropriate solvent with one or more of the various other ingredients described above, followed by sterilization. Dispersions may generally be prepared by incorporating the various sterilized active ingredients into a sterile vehicle that contains the basic dispersion medium and the required other ingredients from those listed above. In the case of sterile powders used to prepare sterile, injectable solutions, the preferred methods of preparation are vacuum-drying and freeze-drying techniques which yield a powder of the active ingredient plus any additional desired ingredient from previously sterile-filtered solutions. Pharmaceutical formulations for topical administration may be especially useful for localized treatment. Formulations for topical treatment included ointments, sprays, gels, suspensions, lotions, creams, and the like. Formulations for topical administration may include known carrier materials such as isopropanol, glycerol, paraffin, stearyl alcohol, polyethylene glycol, and the like. The pharmaceutically acceptable carrier may also include a known chemical absorption promoter. Examples of absorption promoters are e.g., dimethylacetamide (U.S. Pat. No. 3,472,931), trichloroethanol or trifluoroethanol (U.S. Pat. No. 3,891,757), certain alcohols and mixtures thereof (British Patent No. 1,001,949), and British Patent No. 1,464,975. Except insofar as any conventional media or agent is incompatible with the therapeutic active ingredients, its use in the therapeutic compositions and preparations is contemplated.

Supplementary active ingredients can also be incorporated into the compositions and preparations. For example, administration of SPP, its salts and derivatives, and analogs of SPP, their salts and derivatives in combination with other angiogenic factors (such as FGF and/or VEGF) is expected to maximally stimulate angiogenesis.

The compositions and preparations described preferably contain at least 0.1% of active agent. The percentage of the compositions and preparations may, of course, be varied, and may contain between about 2% and 60% of the weight of the amount administered. The amount of active compounds in such pharmaceutically useful compositions and preparations is such that a suitable dosage will be obtained.

Still another embodiment of the present invention comprise inhibition of the expression of SPP receptors such as EDG-1 and EDG-3 by the administration of an effective quantity of a pharmaceutically effective antisense oligonucleotide construct for the expression of either EDG-1 or EDG-3. “Antisense” as used herein refers to nucleotide sequences that are complementary to a specific DNA or RNA sequence. Antisense sequences may be produced by any method, including chemical synthesis, or by ligating the nucleotide sequence of interest in a reverse orientation to a promoter that permits the synthesis of a complementary strand. Once the antisense strand is introduced into a cell, it combines with the natural sequences produced by the cell to form duplexes. These duplexes then block either the further transcription or translation of the gene.

A series of 18-mer phosphothioate oligonucleotides (PTO) were synthesized as potential antisense blocking agents to inhibit the expression of EDG-1 and EDG-3 receptors (FIG. 12). The PTOs are designed to bind to the translational initiation site on the mRNA of the EDG-1, -3, and -5 receptors. Sequences represented by SEQ ID NO:3and SEQ ID NO:6 are the sense sequences for EDG-1 and EDG-3, respectively. Sequences represented by SEQ ID NO: 1 and SEQ ID NO:2 are antisense sequences for EDG-1, wherein the start points differ by three bases. The sequence represented by SEQ ID NO:5 is an antisense sequence for EDG-3. The sequence represented by SEQ ID NO:8 is an antisense sequence for EDG-5. Sequences represented by SEQ ID NO:4 and SEQ ID NO:7 are the “scramble” control sequences for EDG-1 and EDG-3, respectively.

The specificity and efficacy of the PTOs were tested in Xenopus oocytes programmed to express EDG-1 and EDG-3 receptors. Coinjection of either EDG-1 antisense PTO with the EDG-1 cRNA resulted in profound inhibition of EDG-1 expression as determined by suppression of SPP-induced calcium rises. EDG-3 antisense PTO did not inhibit the EDG-1 cRNA for EDG-3. Likewise, EDG-3 antisense PTO only inhibited the cRNA for EDG-3. These data suggest that the EDG-1 and -3 PTOs are specific inhibitors of respective receptor expression. Similar results were obtained upon injection into the cytoplasm of HUVEC. Neither the complementary not the scrambled sequences of EDG-1 and EDG-3 oligonucleotides inhibited VE-cadherin assembly significantly.

Administration of these antisense constructs or their analogs can be used to inhibit angiogenesis in vivo, for example in the neovascularization of tumor cells or other pathological conditions such as rheumatoid arthritis, diabetic retinopathy, Kaposi's sarcoma, hemangioma, and/or psoriasis. The oligonucleotides may be adapted or formulated for administration to the body in a number of ways suitable for the selected method of administration, including orally, intravenously, intramuscularly, intraperitoneally, topically, and the like. In addition to comprising one or more different oligonucleotides, the subject pharmaceutical oligonucleotide formulations may comprise one or more non-biologically active compounds, i.e., excipients, such as stabilizers (to promote long term storage), emulsifiers, binding agents, thickening agents, salts, preservatives, and the like. Delivery of oligonucleotides as described herein is well known in the art for a wide range of animals, including mammals, and especially including humans. For example, Delivery Strategies for Antisense Oligonucleotide Therapeutics, CRC press (Saghir Akhtar, ed. 1995) details many such delivery routes and strategies. By way of example only, and without limiting the applicability of the entire reference, Chapter 5 describes administration by traditional intravenous, intraperitoneal and subcutaneous routes, along with “non-damaging routes” such as intranasal, ocular, transdermal andiontophoresis routes, all of which are applicable to the present invention.

Chapter 6 of the same reference deals with modifications to make oligonucleotide pharmaceuticals nuclease resistant, and the terms nucleotide(s), oligonucleotide(s) and nucleic acid base(s) as used herein specifically includes the described modifications and all other conservatively modified variants of the natural form of such compounds. Modified oligonucleotides, including backbone and/or sugar modified nucleotides as set forth in U.S. Pat. No. 5,681,940, may be used advantageously to enhance survivability of the oligonucleotides.

The claimed oligonucleotides can also be bonded to a lipid or other compound actively transported across a cell membrane, either with or without a linker, and administered orally as disclosed in U.S. Pat. No. 5,411,947, which is also incorporated herein by reference. Still further, the oligonucleotides can be administered in a “naked” form, encapsulated, in association with vesicles, liposomes, beads, micro spheres, as conjugates, and as an aerosol directly to the lung, using for example ICN Biomedicals product no. SPAG 2. Thus, the described oligonucleotides can be administered substantially by all known routes of administration for oligonucleotides, using all accepted modifications to produce nucleotide analogs and prodrugs, and including all appropriate binders and excipients, dosage forms and treatment regimens.

The oligonucleotides are administered in dosages and amounts that are conventional in the art for the underlying bioactive compound, but adjusted for more efficient absorption, transport and cellular uptake. The dosages may be administered all at once, or may be divided into a number of smaller doses, which are then administered at varying intervals of time. The specific treatment regimen given to any individual patient is readily determined by one of ordinary skill in the art, and will, of course, depend upon the experience of the clinician in weighing the disease involved, the health and responsiveness of the patient, side effects, and many other factors as is well known among such clinicians. Standard treatment regimens comprise intravenous administration of between about 0.1 and 100 mg of oligonucleotide per kilogram of body weight of the patient, 1-14 times per week for approximately 40 days.

For oral administration, the oligonucleotides may be formulated as described above in connection with SPP and SPP analogs. Solutions of the oligonucleotides may be stored and/or administered as freebase or pharmacologically acceptable salts, and may advantageously be prepared in water suitably mixed with a surfactant such as hydroxypropylcellulose. Dispersions can also be prepares in glycerol, liquid polyethylene glycols, and mixtures thereof and in oils.

In addition to the therapeutic uses of the subject oligonucleotides, the oligonucleotides may also be used as the laboratory tool for the study of absorption, distribution, cellular uptake, and efficacy.

In still another embodiment, a gene therapy method comprises construction and administration of vectors effective to overexpress EDG-1 and EDG-3 in the endothelial cells of the body in an amount effective to induce angiogenesis. For example, the EDG-1 and -3 cDNAs can be expressed using the pCDNA vector (Invitrogen) which contains the cytomegalovirus promoter (CMV) for high-level expression in endothelial cells. In addition, adenoviral vectors containing the CMV promoter or endothelial cell-specific TIE II promoter can be used to express the EDG-1 and -3 cDNAs as well.

In yet another embodiment, a gene therapy method comprises construction and administration of vectors effective to inhibit expression of EDG-1 and EDG-3 in the endothelial cells of the body in amount effective to inhibit angiogenesis. A construct containing the EDG-1 and -3 cDNAs in antisense orientation and controlled by the cytomegalovirus promoter can be used to express EDG-1 and -3 antisense cDNAs in endothelial cells to inhibit the expression of respective receptors.

Yet another embodiment of the invention relates to the discovery that the EDG-1 receptor is phosphorylated by the Akt protein kinase. Many angiogenic factors such as vascular endothelial cell growth factor (VEGF) utilize the PI-3 kinase/Akt signaling pathway to regulate endothelial cell behavior important in angiogenesis such as cell migration and survival. Furthermore, activation of EDG-1 regulates intracellular signaling pathways, resulting in endothelial cell migration. It has been found that phosphorylation of the EDG-1 receptor by the protein kinase Akt is critical for cells to commit to chemotaxis.

It was noticed that the third intracellular loop of EDG-1 contains a consensus Akt phosphorylation site. Immunoprecipitation experiments show that Akt associates with EDG-1 and also phosphorylates EDG-1. The phosphorylation site was identified as T²³⁶, a site within the known Akt consensus. Because Sphingosine 1-phosphate (SPP) activates EDG-1, the effects of SPP on Akt phosphorylation in HUVEC cells were determined. SSP was shown to induce phosphorylation of Akt through a Gi/PI-3-kinase pathway using Wortmannin and LY294002 as PI-3 kinase inhibitors. SPP was further shown to stimulate the association of Akt and EDG-1 and to stimulate phosphorylation of EDG-1. The PI-3 kinase inhibitor LY294002 suppressed the SPP-induced phosphorylation of EDG-1. To determine if SPP induces phosphorylation of endogenous EDG-1, a chicken anti-human EDG-1 antibody was developed. Treatment with SPP increased the amount of phosphorylated EDG-1 and this phosphorylation was inhibited by Wortmannin and LY294002. Together, these results show that activation of Akt results in is association with and phosphorylation of EDG-1.

Since Akt is a known intermediate in chemotaxis, the role of Akt activity in SPP-induced responses in HUVEC cells was determined. Adenoviral constructs containing wild type Akt, dominant-negative Akt and constitutively active Akt were transduced in HUVEC calls. The wild type Akt induced weak cortical fibers, the constitutively active Akt induced strong cortical actin structures, while the dominant negative Akt blocked SPP-induced cortical actin fibers. The SSP-induced cortical actin structures were inhibited by the PI-3 kinase inhibitors Wortmannin and LY294002. The role of Akt signaling in cell migration was also studied in CHO cells overexpressing the EDG-1 and EDG-3 receptors. SSP induced cell migration in CHO cells expressing EDG-1 but not EDG-3. Taken together, all of these observations show that the Akt signaling pathway is required for formation of EDG-1-induced cortical actin structures.

To further define the function of Akt phosphorylation, the phosphorylated T²³⁶ residue in EDG-1 was mutated to A²³⁶. When mRNA encoding wild-type or T236AEDG-1 together with the heterotrimeric G₁ protein was expressed in Xenopus oocytes and the oocytes were stimulated with S1P, intracellular calcium rises were observed, suggesting that the T236A mutation does not impair coupling to the G₁ pathway. Also, the T236AEG-1 mutant receptor associated with Akt, similar to the wild-type EDG-1. Thus, a mutant EDG-1 receptor which cannot be phosphorylated by Akt still associates with Akt and can participate in rapid signal transduction events such as intracellular calcium rises. Further mutants in the Akt consensus R231K and R233K were prepared. These mutants are also poorly phosphorylated by Akt in vitro, although they can be phosphorylated by other kinases such as P90^{RSK}. Transfected CHO cells with T236A, R231K or R233K EDG-1 receptors fail to respond to added SPP and do not induce CHO cell migration. Further experiments showed that the T236A EDG-1 receptor cannot activate the Rac GTPase thus blocking cortical actin assembly and chemotaxis. The T236A receptor acts as functionally as a dominant negative G-protein coupled receptor by sequestering the Akt. The T236A mutant further blocks angiogenesis in vitro and in vivo using matrigel plugs in an in vitro HUVEC cell model and an in vivo nude mouse model.

The invention is further illustrated by the following non-limiting examples. Many of the techniques discussed herein, including, for example, conditions for stringency of hybridization, are more fully described in laboratory manuals such as ‘Molecular Cloning: A Laboratory Manual’ Second Edition by Sambrook et al., Cold Spring Harbor Press, 1989.

### EXAMPLES

### Example 1

### Expression of EDG mRNA in Endothelial Cells

Human umbilical vein endothelial cells (HUVEC) (cell line Cc-2517; Clonetics Corporation, Walkersville, Md.) were cultured in M199 medium (Mediatech, Inc., Herndon, Va.) supplemented with 10% fetal bovine serum (FBS, HyClone Laboratories, Inc., Logan, Utah) and heparin-stabilized endothelial cell growth factor, as described previously (Hla, T., and Maciag, T., J. Biol. Chem. 265: 9308-9313 (1990). HUVEC from passage numbers 4-12 were used. Human Embryonic Kidney 293 (HEK293) cells (cell line ATCC CRL-1573, American Type Culture Collection, Manassas, Va.) and RH7777 rat hepatoma cells (Zhang et al., Gene 227: 89-99 (1999)) were cultured in Dulbecco's Modified Eagle's Medium (DMEM) containing 10% fetal bovine serum (FBS). Cells were harvested, and poly(A)⁺ was isolated from the HUVEC and from the HEK293 cells. In vitro transcripts of EDG-1, EDG-3, and EDG-5 were prepared as described (Zhang et al., Gene 227: 89-99 (1999)). Two μg of HUVEC and HEK293 poly (A)⁺ RNA, 20 μg of rat hepatoma total RNA and 280 pg of the EDG-1, EDG-3, EDG-5 in vitro transcripts were loaded and separated on a 1% agarose gel, then transferred overnight to a Zeta Probe Blotting Membrane (Bio-Rad Laboratories Inc., Hercules, Calif.). Probes were prepared with the Random Primed DNA Labeling Kit (Boehringer Mannheim, now Roche Diagnostics, Indianapolis, Ind.) using the following open reading from DNA templates: mouse EDG-1920 bp fragment, human EDG-3 1.1 kb fragment, rat EDG-5 1.1 kb fragment and human GAPDH 600 bp fragment. Northern analysis was performed as descried by Lee, M. J., et al., J. Biol. Chem. 273: 22105-22112 (1998).

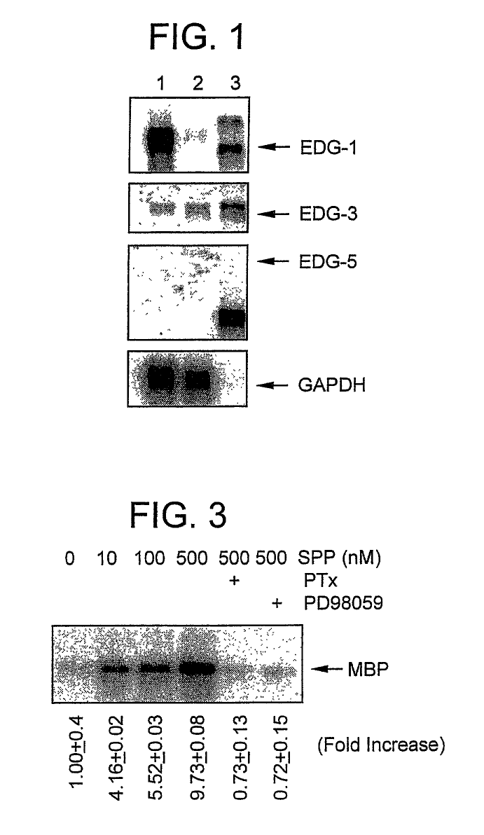

FIG. 1 shows results from Northern blots of RNA obtained from the above sources, wherein poly(A)+ RNA from HUVEC (lane 1) and HEK293 (lane 2) were probed with EDG-1, EDG-3, EDG-5, or GAPDH (control) cDNAs. In vitro transcripts for EDG-1, -3, and -5 are also shown as positive controls (+VE, lane 3). EDG-1 mRNA was abundantly expressed, but only a small amount of the EDG-3 mRNA was detected, and EDG-5 mRNA was not detected. EDG-1 expression was estimated to be 16 fold more abundant than the EDG-3 signal in HUVEC as determined by phosphoimager analysis. In contrast, EDG-3 is the predominant SPP receptor isotype in HEK293 cells. Total RNA preparations for RH7777 hepatoma cells contain transcripts for both EDG-1 and EDG-5 isoforms. EDG-1 is therefore the most abundant EDG transcript detected in endothelial cells.

### Example 2

### Determination of G Protein-Coupled Receptors for SPP in Endothelial Cells

Functional assays were used to test for the presence of Gᵢ-coupled and G_{q}-coupled SPP receptors in HUVEC. First, intracellular calcium levels were measured in response to SPP. For these experiments, cells were grown on 100-mm tissue culture dishes and loaded with the fluorescent calcium-sensitive dye, Indo-1 acetoxymethyl ester (Indo-1/AM, 5 μg/mL; Molecular Probes, Inc., Eugene, Oreg.), for 30 min at 37° C. Cells were then washed with medium M199, briefly trypsinized (0.05% porcine trypsin/0.02% EDTA in HBSS (JRH Biosciences, Lenexa, Kans.), and the trypsin activity was immediately neutralized with soybean trypsin inhibitor (5 μg/mL, Sigma Chemical Co., St. Louis, Mo.). Following centrifugation (250 g×5 min), cells were resuspended in M199 medium to a density of 2.7×10⁵ cells/mL. Cells were then stimulated with different doses of SPP. Some cells were pretreated with the Gᵢ inhibitor pertussis toxin (PTx, 500 ng-/mL) for 16 hours. Calcium ion concentration was then quantified by measuring changes in indo-1 fluorescence in 2 mL of cell suspension by a Hitachi F-2000 fluorescence Spectrophotometer with constant stirring. Fluorescence emission was monitored at 400 and 475 nm with excitation at 352 nm. [Ca⁺²]₁ was calculated as described in Volpi and Berlin, J. Cell Biol., Vol. 107, 2533-39 (1988).

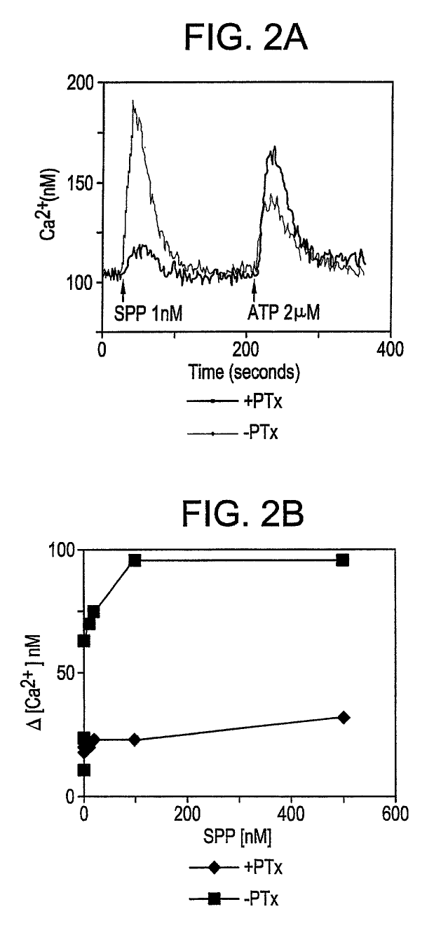

As shown in FIGS. 2A and 2B, SPP induced a robust calcium response in endothelial cells. The SPP-induced response was inhibited approximately 90% by treatment with pertussis toxin whereas the G_{q}-coupled ATP receptor response was not pertussis toxin sensitive. These data suggest that EDG-1, a Gᵢ-coupled SPP receptor, is responsible for most of SPP-induced extracellular signal-activated kinase (ERK) activation assay.

As a second functional assay, ERK-2 kinase activity was measured in response to SPP treatment. For these experiments, endothelial cells were starved for 19 hours, and then stimulated with SPP for 10 minutes. Cells were then lysed, and ERK-2 kinase activity was measured by an immune complex kinase assay using myelin basic protein (MBP) as substrate. Some cells were pretreated with pertussis toxin (PTx) at 200 ng/mL, or PD98059 at 10 μM for 2 hours prior to stimulation. As shown in FIG. 3, SPP (10-500 nM) activated ERK activity in a dose-dependent manner in HUVEC. This response was inhibited completely by pretreating cells with pertussis toxin and PD98059, indicating that this activity is dependent on the Gᵢ protein and MAP kinase. Complete inhibition by pertussis toxin suggests that most of the SPP effects are mediated by the Gᵢ-coupled SPP receptor, EDG-1.

### Example 3

### Rho- and Rac-Dependent Cytoskeletal Changes Induced by SPP in Endothelial Cells

SPP is known to induce Rho-dependent actin stress fibers in NIH3T3 fibroblasts. To determine Rho- or Rac-dependence, HUVEC were plated at 2×10⁵ cells in 35 mm glass bottom petri dishes (Plastek cultureware, Mat Tek Corporation, Ashland, Mass.). Two days later, cells were washed and changed to medium M199 supplemented with 10% dialyzed CFBS and growth factors for 16 hours. Approximately 500-800 cells were then microinjected cytoplasmically with Rho inhibit C3 exoenzyme (0.1 μg/μl, Calbiochem), or dominant negative N17Rac protein (0.35 μg/uL; Ridley, A. et al., Cell, Vol. 70, 401-410 (1992) using Femtotips (Eppendorf) at 100 hPa/0.2 sec. Injected cells were marked by coinjection of FITC-rabbit IgG (5 mg/mL, Cappel). Subsequently, cells were treated with or without SPP. After treatment, cells were washed with ice-cold PBS, fixed with 4% formaldehyde at room temperature or methanol at −20° C. for 15 minutes. In the case of formaldehyde fixation, cells were permeabilized with 0.2% Triton-X 100. After washing with PBS, cells were stained with various antibodies as follows: VE-cadherin (1.25 μg/mL, Transduction Labs, San Diego, Calif.; 1 μg/mL, Santa Cruz Biotechnology, Inc., Santa Cruz, Calif.), beta-catenin (1.25 μg /mL, Transduction Labs); gamma-catenin (1.25 μg /mL, Transduction Labs), alpha-catenin(1.25 μg /mL, Transduction Labs), Tiam1 (1 μg/mL, Santa Cruz), Rac (1 μg/mL, Santa Cruz), Rho (0.4 μg/mL, Santa Cruz). The primary antibody staining was visualized with FITC conjugated goat anti-rabbit or TRITC conjugated sheep anti-mouse (1:1000, Cappel, now owned by ICN, Costa Mesa, Calif.) IgG for 30 minutes at room temperature.

### Example 4

### Rho- and Rac-Dependent Cytoskeleton Reorganization in Endothelial Cells

SPP is known to induce Rho-dependent actin stress fibers in NIH3T3 fibroblasts. To disclose Rho- or Rac-dependence, HUVEC were plated at 2×10⁵ cells in 35 mm glass bottom petri dishes (Plastek cultureware, Mat Tek Corporation, Ashland Mass.). Two days later, recently confluent cells were washed and changed to medium M199 supplemented with 10% dialyzed charcoal-stripped fetal bovine serum (CFBS) and growth factors for 16 hours. Approximately 500-800 cells were then microinjected cytoplasmically with Rho inhibitor C3 exoenzyme (0.1 μg/μL, Calbiochem) or dominant negative N17Rac protein (0.35 μg/μl; Ridley, A., et al., Cell 70: 401-410 (1992)) using Femtotips (Eppendorf) at 100 hPa/0.2 sec. Injected cells were marked by coinjection of FITC-rabbit IgG (5 mg/mL, Cappel).

Subsequently, cells were treated with or without SPP. After treatment cells were washed with ice-cold PBS, fixed with 4% formaldehyde at room temperature or methanol at −20° C. for 15 minutes. In the case of formaldehyde fixation, cells were permeabilized with 0.2% Triton X-100 (TX-100). Actin microfilaments were visualized by staining with either FTOC- or TRITC-conjugated phalloidin (0.05 μg/mL, Sigma) for 30 minutes at room temperature.

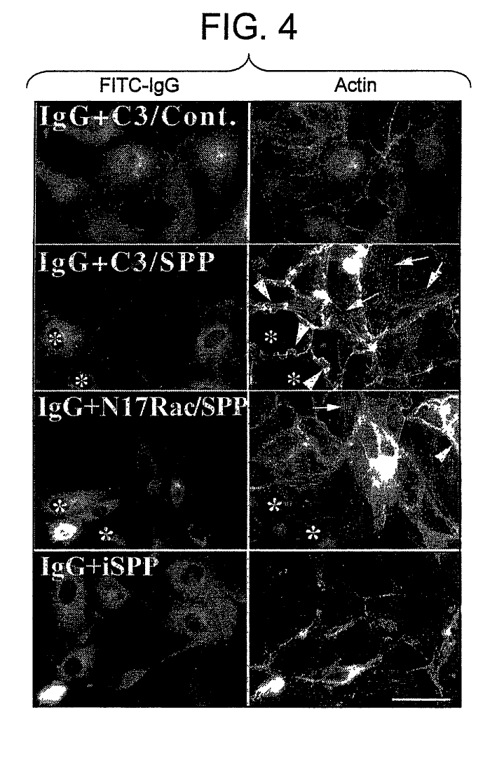

As shown in FIG. 4, intracellular microinjection of HUVEC with the C3 exoenzyme abolished with SPP-induced stress fibers after 2 hours. (FIG. 4, first and second rows). In contrast, C3 exoenzyme microinjection did not block SPP-induced cortical actin formation (FIG. 4, first and second rows). However, microinjection of dominate negative Rac protein N17Rac for 2 hours completely inhibited the formation of both stress fibers and cortical actin (FIG. 4, third row). Microinjection of control rabbit IgG did not inhibit SPP-induced stress fiber and cortical actin assembly (data not shown). Cells were stimulated without (first row) or with (second and third rows) 500 nM SPP for 30 minutes. Injected cells were marked with FITC-rabbit IgG (left column). SPP induced the formation of stress fibers (arrows) and cortical actin (arrowheads). iSPP=direct intracellular microinjection of SPP (500 μM). These data suggest that the extracellular action of SPP transduces signals via the Rac and Rho small GTPascs to regulate the cytoskeletal architecture of endothelial cells. Furthermore, Rac appears to act upstream of Rho in cytoskeletal changes.

### Example 5

### SPP Regulates Adherens Junction Assembly in HUVEC

SPP has been demonstrated to induce morphogenetic differentiation and upregulate P-cadherin levels in FDG-1-transfected HEK293 cells. To investigate whether SPP regulates the formation of adherens junction in endothelial cells, HUVEC were plated at a density of 2×10⁴ cells/em² for 2 days, starved in lipid-depleted medium and treated without (cont) or with SPP (500 nM for 1 hours). For these experiments, after treatment cells were washed with ice-cold PBS, fixed with 4% formaldehyde at room temperature or methanol at −20° C. for 15 minutes. In the case of formaldehyde fixation, cells were permeabilized with 0.2% TX-100. After washing with PBS, cells were stained with antibodies as follows: VE-cadherin (1.25 mg/mL, Transduction Labs, San Diego, Calif.; 1 μg/mL, Santa Cruz Biotechnology, Inc., Santa Cruz, Calif.), α-catenin (1.25 mg/mL, Transduction Labs), β-catenin (1.25 μg/mL, Transduction Labs; 0.4 μg/mL, Santa Cruz), γ-catenin (1.25 mg/mL, Transduction Labs). The primary antibody staining was visualized with FITC-conjugated goat anti-rabbit or TRITC conjugated sheep anti-mouse (1:1000, Cappel, now owned by ICN, Costa Mesa, Calif.) for 30 minutes at room temperature, and imaged on a Zeiss Axiovert 100TV fluorescence microscope.

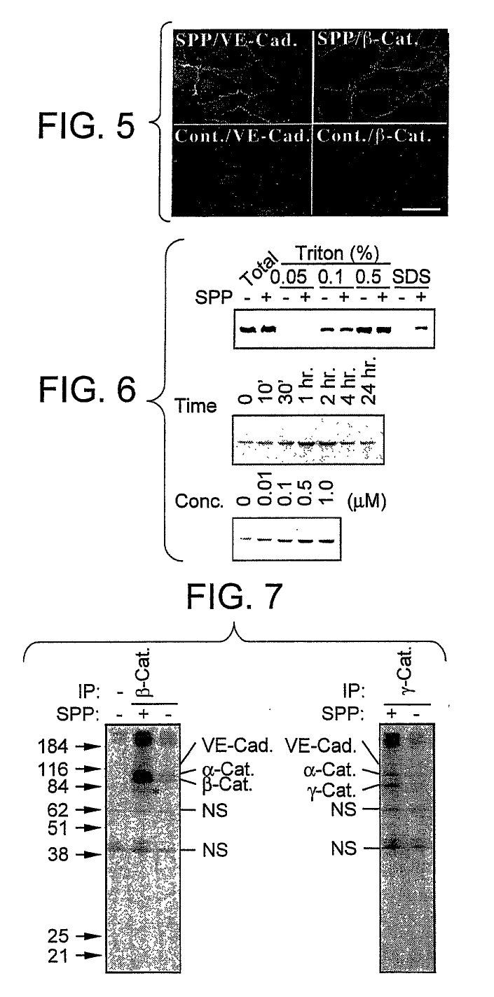

As shown in FIG. 5 for VE-cadherin and β-catenin, within one hour following SPP treatment, VE-cadherin α, β- and γ-catenin localization at cell-cell junctions were dramatically increased. Confocal immunofluorescence microscopy indicated that SPP treatment increased the localization of VE-cadherin (FIG. 5) into discontinuous structures at cell-cell contact regions, suggested that SPP induces the formation of adherens junctions. Scale bar represents 13.4 microns. Treatment with related lipids which do not interact with SPP receptors (sphingosine, sphingomyelin, ceramide, ceramide-1-phosphate) had no effect.

### Example 6

### SPP Induces Formation of Triton X-100 Insoluble VE-Cadherin

To investigate whether SPP induces TX-100 insolubility of VE-cadherin, unstimulated HUVEC (−) or HUVEC stimulated with 500 nM SPP for 1 hour (+) were sequentially fractionated with TX-100 (0.05, 0.1, 0.5%) (FIG. 6). For these experiments, HUVEC were fractionated with cytoskeleton stabilizing buffer (10 mM 1`1EPES, p117.4, 250 mM sucrose, 150 mM KCl, 1 mM EGTA. 3 mM MgCl₂, 1× protease inhibitor cocktail (Calbiochem), 1 mM Na₃VO₄. Following centrifugation (15,000 g, 15 minutes.), the detergent-soluble and -insoluble fractions were separated. The detergent-insoluble fractions were extracted with 1% Tx-100-1% SDS in cytoskeleton stabilizing buffer at 95° C. for 10 min. Equal amount of protein extracts were loaded and probed with anti-VE-cadherin antibody (upper panel). HUVEC were stimulated with 500 nM SPP for inducated times, then extracted with 0.5% TX-100. Insoluble fractions were further extracted with 1% TX-100 plus 1% SDS, and probed for VE-cadherin in a Western blot (middle panel). HUVEC were treated for 1 hour with indicated concentration of SPP.TX-100-resistant VE-cadherin levels were determined as described above (lower panel). As shown in FIG. 6, fractionation of HUVEC cell lysates into Triton-X-100-soluble and -insoluble fractions showed that SPP induced an increase in the amount of VE-cadherin in the TX-100 insoluble fraction; however, the overall level of protein was not altered. SPP induced increase of VE-cadherin in the Triton X-100 insoluble fraction was dose-and time-dependent. Consistent with the immunofluorescence data, the increase of VE-cadherin in Triton X-100 insoluble fractions peaked at 1-2 hours following SPP treatment.

To directly show that SPP signaling in endothelial cells regulates adherens junction assembly, a co-immunoprecipitation experiment was conducted. HUVEC were labeled to steady state with ^{35S}methionine (250 μCi/mL, NEN DuPont) for 24 hours. After stimulation with 500 nM SPP for 1 hour, cells were fractionated with 0.5% TX-100. After centrifugation (15,000 g, 15 minutes), the protein complexes in detergent-insoluble fractions were cross-linked with 0.5 mM Dithiobis[succinimidyl propionate] (DSP; Pierce Chemical Co., Rockford, Ill.) (Hinck, L., J. Cell Biol. 125: 1327-1340 (1994)), and extracted with 1% TX-100-1% SDS as described above. Cell extracts were then immunoprecipitated with antibodies to VE cadherin, β-catenin, γ-catenin, or p120 Src (p120^{cas}, Transduction Laboratories). The immunoprecipitated complexes were then reduced by incubating in sample buffer containing 2% β-mercaptoethanol at 95° C. for 10 minutes, 10 μL dithiothreitol (1 M) was added to each gel lanes before protein separation by SDS-PAGE.

As shown in FIG. 7, SPP significantly increased the catenin and VE-Cadherin polypeptides in the β- and γ-catenin immunoprecipitates. An unidentified polypeptide (*) of about 80 Kd was also co-immunoprecipitated in a SPP-sensitive manner. In agreement with previous findings, β-catenin and γ-catenin are found in a mutually exclusive manner in endothelial cell adherens junction complexes.

### Example 7

### SPP-Induced Adherens Junction Assembly Requires Rho and Rac Small GTPases

In order to probe the relationship between SPP treatment and subcellular localization of Rac and Rho small GTPases, immunofluorescence microscopy before and after SPP treatment of HUVEC was conducted. In these experiments, recently confluent cells were stimulated with 500 nM SPP for 30 minutes, immunostained with antibodies against Rac (1 μg/mL, Santa Cruz) Rho (0.4 μg/mL, Santa Cruz), and/or the Rho-specific guanine nucleotide exchange factor Tiam 1(1 μg/mL, Santa Cruz). Primary antibody binding was revealed using FITC-conjugated goat anti-rabbit and/or TRITC-conjugated sheep anti-mouse (1:1000, Cappel) as described above.

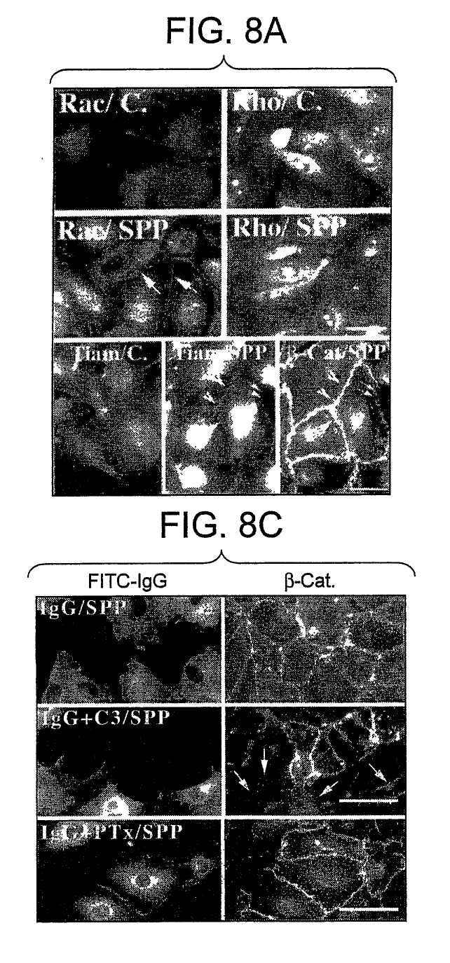

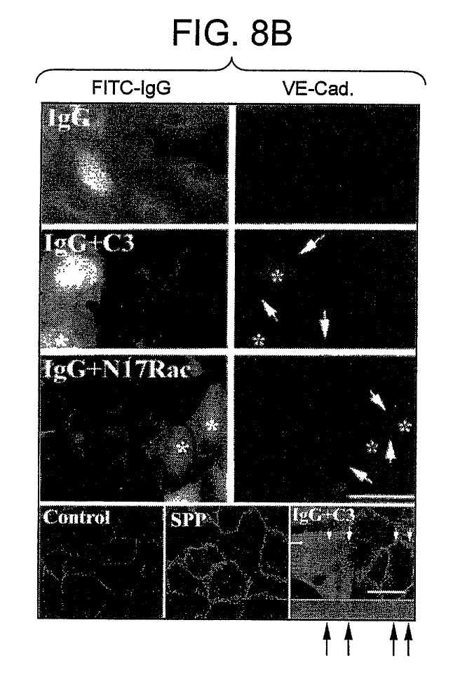

As shown in FIG. 8A, SPP induces the translocation of Rac and Tiam 1 to cell-cell contact sites. The anti-Rac antibody specifically reacts with fine dot-like structures, which are evenly distributed throughout the cytoplasm. However, treatment with SPP for 10-30 minutes resulted in significant redistribution of Rac to the cell-cell contact areas. In contrast, subcellular localization of Rho as not altered after SPP treatment. Tiam 1 also translocated to cell-cell contact areas as a result of SPP treatment. Double immunostaining demonstrated an overlapping pattern between Tiam 1 and β-catenin after SPP treatment. These data suggest that SPP signaling activates the translocation of Tiam 1 and Rac to the cell-cell contact areas to regulate VE-cadherin assembly into adherens junctions. Scale bar in upper panels represents 8.7 microns. Scale bar in lower panels represents 7.7 μM. Rac/C., Rho/C., and Tiam/C. are unstimulated cells labeled with antibodies against Rac, Rho, and Tiam 1, respectively; whereas Rac/SPP, Rho/SPP, Tiam 1/SPP, and β-Cat/SPP are SPP-stimulated cells labeled with antibodies against Rac, Rho, Tiam 1 and β-catenin, respectively.

To determine if Rho and Rac small GTPases are required for SPP induced adherens junction assembly, C3 exoenzyme and dominant negative N17Rac polypeptide were microinjected in HUVEC cells. As shown in FIG. 8B, microinjection of C3 or N17Rac dramatically diminished SPP-induced VE-cadherin and β-catenin immunoreactivity at cell-cell junctions. Following stimulation with SPP-induced VE-cadherin and β-catenin immunoreactivity at cell-cell junctions. Following stimulation with SPP, cells were stained with anti-VE cadherin. HUVEC were microinjected with FITC-IgG alone (first row); FITC-IgG together with C3 exoenzyme (second row) or N17Rac (third row). Following stimulation with SPP, cells were stained with an antibody against VE-cadherin. Arrows in FIG. 8B indicate contact areas between cells injected with C3 or N17Rac exhibiting diminished SPP-induced VE-cadherin immunoreactivity. Scale bar indicates 20 μM. Lower panels show confocal images of anti-Ve-cadherin staining in unstimulated (left) or SPP-stimulated (middle) HUVEC. When cells were injected with FITC-IgG plus C3 exoenzyme and stained with anti-VE-cadherin, superimposed confocal image (right) show that SPP-induced zigzag-like staining pattern was reduced to a fine line by C3 treatment (FITC-positive cells). Inset shows the Z-section of the confocal image. Strong VE-cadherin staining was observed in the apical region of cell-cell junctions in uninjected cells (vertical arrows), whereas only a weakly stained smooth line was observed in the apical region of cell-cell junctions in uninjected cells (vertical arrowhead). The position of the Z-section is indicated by a horizontal arrow. Scale bar indicates 11.25 microns. Confocal microscopy was carried out as described elsewhere (Liu, C., et al., Mol. Biol. Cell 10: 1179-1190 (1999)).

In order to investigate if β-catenin translocation induced by SPP treatment requires Rho activity, cells were microinjected with C3 exoenzyme or Pertussis toxin (1 μg/mL). Each injection also included FITC-IgG. As shown in FIG. 8C, injection of C3 exoenzyme but not PTx significantly inhibited the translocation of β-catenin polypeptide. Upper scale bar=22.4 microns (for first and second rows), lower scale bar=15.3 microns (for third row).

### Example 8

### EDG-1 and EDG-3 Mediate SPP-Induced Morphogenesis and Survival

To investigate SPP induction of angiogenesis, 200 μL aliquots of thawed MATRIGEL were polymerized in 24-well tissue culture plates. HUVEC were trypsinized, resuspended in plain M199 medium containing soybean trypsin inhibitor (10 mg/mL, Sigma Chemical Co., St. Louis, Mo.). Following centrifugation (250 g; 5 minutes), cells were resuspended in plain M199 supplemented with 2% CFBS at a density of 1.5×10⁵ cells/mL. 200 μL of cell suspension were seeded on these gels in the presence or absence of SPP (Biomol), for 12-18 hours. Cells were rinsed two times with phosphate buffered saline (PBS), and then fixed with 4% formaldehyde. Results were recorded photographically using a Zeiss Axiovert 100TV microscope equipped with a 5× objective. Five random fields of each well were photographed, and total tubular length was quantified by image analysis (Kinsella, J. et al., Experimental Cell Research 199: 56-62 (1992); Gamble J., et al., J. Cell Biol. 121: 931-943 (1993)).

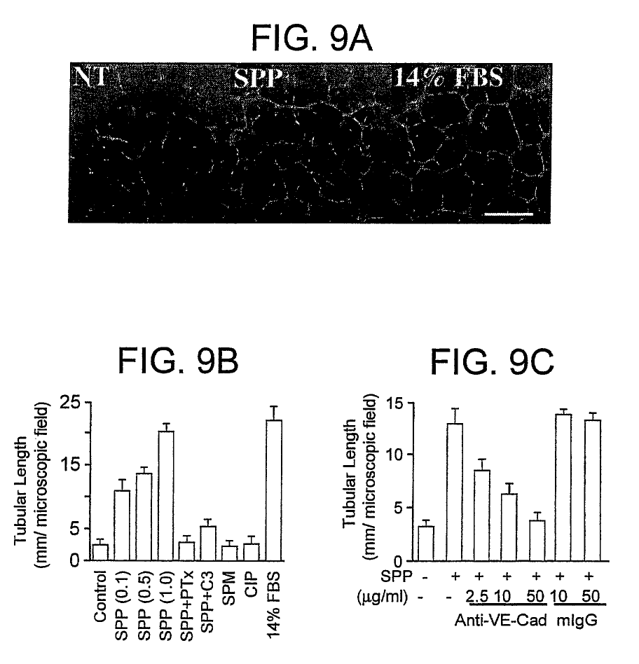

As shown in FIGS. 9A-B, SPP promoted HUVEC morphogenesis in a dose-dependent manner, whereas lipid analogs ceramide-1 phosphate and sphingomyelin, which do not activate EDG-1, were inactive. FIG. 9A shows morphogenesis on MATRIGEL, whereas FIG. 9B presents a quantitative analysis of tubular length. Scale bar represents 52 microns. These quantitative data are the mean±standard deviation of duplicate determinations from a representative experiment which was repeated at least three times. SPP concentrations (in μM) are indicated in parentheses. For SPP +PTx, HUVEC were pretreated with PTx (200 ng/mL) for 2 hours, trypsinized, plated onto MATRIGEL, then stimulated with 500 nM SPP in the presence of PTx (20 ng/mL) for 16-18 hours. For SPP+C3, HUVEC were pretreated with C3 exoenzyme (10 μg/mL) for 48 hours, trypsinized, plated onto MATRIGEL, stimulated with 500 nM SPP together with the same concentration of C3 exotoxin. SPM, sphingomyelin (1 μM). For C1P, C8-ceramide-1-phosphate (1 μM). The maximal effects achieved by 1 μM SPP was indistinguishable from the positive control medium which contained FBS. Also, SPP, ranging from 100 nM to 1 μM, induced morphogenesis of bovine microvascular endothelial cells (data not shown).

### Example 9

### VE-Cadherin is Required for SPP-Induced Morphogenesis

To disclose a requirement for VE-cadherin in SPP-induced morphogenesis of capillaries in vitro, cultured HUVEC were pretreated with various concentrations of an activity-blocking mouse monoclonal antibody against VE-cadherin, which recognizes the extracellular domain of VE-cadherin polypeptide, or else with an irrelevant mouse IgG (mIgG) for 1 hour. In these experiments, HUVEC were plated onto MATRIGEL in the presence of the same amount of corresponding antibodies without or with 500 nM SPP. 16 hours later, total length of HUVEC networks formed on MATRIGEL was quantified.

As shown in FIG. 9C, anti-VE-cadherin antibody, in a dose-dependent manner, inhibited SPP-induced morphogenesis. This effect was specific since no inhibition was observed with an irrelevant mouse IgG. These data indicate that SPP activation of endothelial cells stimulates two distinct signaling pathways: Gᵢ-mediated endothelial cell survival, and Rho-/Rac-mediated Ve-cadherin assembly into adherens junctions. Both of these signaling pathways are important for endothelial cell morphogenesis into capillary-like networks.

### Example 10

### SPP Protects Cells from Apoptosis via the G₁/MAP Kinase Pathway

The ability of SPP to protect endothelial cells from apoptosis was investigated. In these experiments, HUVEC were treated with 1 μM C₂-Ceramide for 12 hours and apoptosis was measured in the following manner: HUVEC were plated onto coverslips and allowed to grow for 2 days. Cells were washed three times with medium M199, and treated with 1 μM C2-Ceramide (Biomol) for 12 hours in the presence or absence of SPP. Subsequently, cells were fixed with methanol at −20° C. for 5 minutes, air dried, and stained with Hoechst 33258 dye (0.5 μg/mL for 5 minutes; Sigma Chemical Co., St. Louis, Mo.). The apoptotic nuclei were identified with the aid of a Zeiss Axiovert 100TB fluorescence microscope. For quantification, HUVEC were labeled with ^{3H}methyl-thymidine (5 μCi/mL, NEN DuPont) for 24 hours. Following three washes with medium M199, cells were treated with C₂-Ceramide as above. 12 hours later, cells were extracted with lysis buffer (5 mM tris, pH 7.4, 2 mM EDTA, 0.5% TX-100) at 4° C. for 20 minutes. After centrifugation (15,000 g, 20 minutes), the radioactivity present in the supernatant and sediment was measured by liquid scintillation counting. The percentage of DNA fragmentation was determined as ((supernatant cpm)/(supernatant cpm+sediment cpm))×100%.

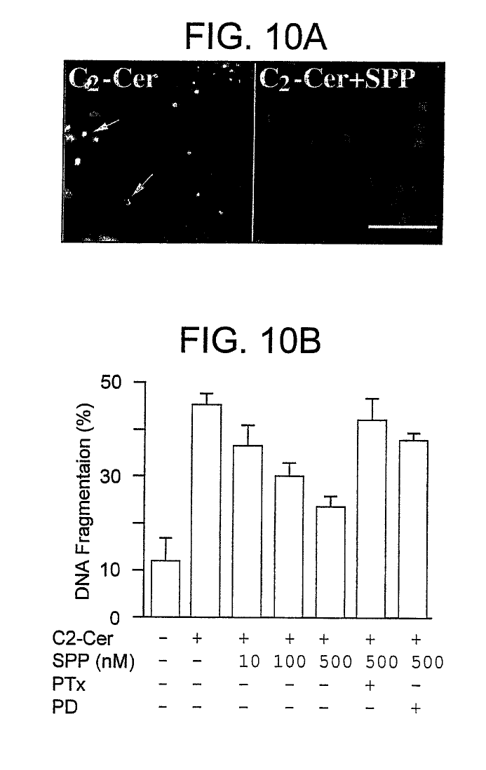

FIG. 10A shows cells treated with C₂-Ceramide in the absence (C₂-Cer, upper panel) or presence (C₂-Cer+SPP, lower panel) of 500 nM SPP. The apoptotic nuclei (arrows in FIG. 10A, upper panel) were identified by staining with the Hoechst dye. The scale bar represents 31 microns. A high percentage of cells are observed to be apoptotic by this assay.

As shown in FIG. 10B, HUVEC were incubated with ^{3H}methyl-thymidine as described above, then washed before exposure to C₂-Ceramide in the presence or absence of SPP for 12 hours. SPP+PTx and SPP+PD98059 (10 μM), respectively, for 2 hours prior to the addition of C₂-Ceramide (1 μM) and SPP (10 to 500 nM). Data are mean±standard deviation of triplicate determinations from a representative experiment which was repeated two times. As can be seen from these data, SPP (10 to 500 nM), significantly protected cells, in a dose-dependent meaner, from apoptosis induced by C₂-Ceramide. A similar effect was also seen when growth factor withdrawal, and 15-deoxy Δ^{12,14} prostaglandin J₂ were used as apoptotic stimuli (data not shown). This cytoprotective effect of SPP is completely inhibited in the presence of pertussis toxin and PD 98059 (FIG. 10B), reagents which inactivate the Gᵢ and MAP kinase, respectively and thus attenuate the ERK signaling pathway. Therefore SPP induced endothelial cell survival requires the Gᵢ/ERK signaling pathway.

Antisense EDG-1 PTO (see example 12) treatment reduced the ability of SPP to block ceramide-induced apoptosis (44±4%) whereas none of sense EDG-1 PTO, antisense EDGE-3 PTO, or antisense EDG-5 PTO had a significant effect (<5%). These data strongly suggest that EDG-1/G/ERK pathway mediates SPP-induced endothelial cell survival.

### Example 11

### Regulation of Angiogenesis by SPP in vivo

To disclose if SPP regulates angiogenesis in vivo, A MATRIGEL implant model of subcutaneous angiogenesis in ethylic mice was used. In these experiments, female ethylic mice (4-6 weeks old) were injected subcutaneously with 0.4 mL MATRIGEL (approximate protein concentration 9.9 mg/mL, Collaborative Research) premixed with vehicle (fatty acid-free BSA, 115 μg/mL, Sigma), or FGF-2 (1.3 μg/mL) in the absence or presence of various concentrations of SPP. Seven days later, MATRIGEL plugs were harvested along with underlying skin and the gross angiogenic response was recorded under a Zeiss Stemi SV6 dissecting microscope. For quantification, MATRIGEL plugs were fixed with 4% paraformaldehyde in PBS, dehydrated in ethanol and xylene, embedded in paraffin, and sections subjected to hematoxylin and eosin staining. Angiogenesis was quantified by direct counting of vessels containing red blood cells residing in the stroma interface and the MATRIGEL implant. Each treatment involved 4 mice. 2 random sections from each were quantified and represented as mean±standard deviation. Transmission electron microscopy of 2.5% glutaraldehyde-fixed MATRIGEL plugs was performed as described (Lee, M., et al., Science 279: 1552-1555 (1998)).

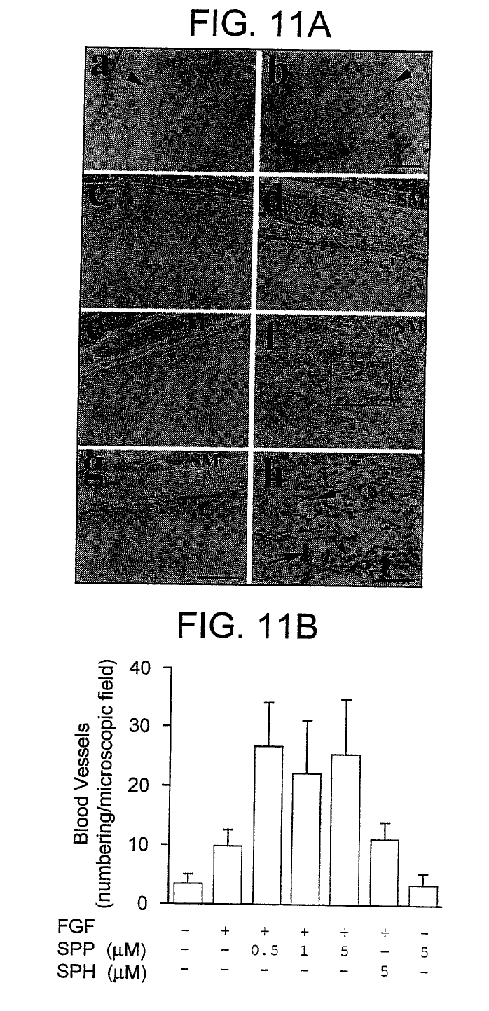

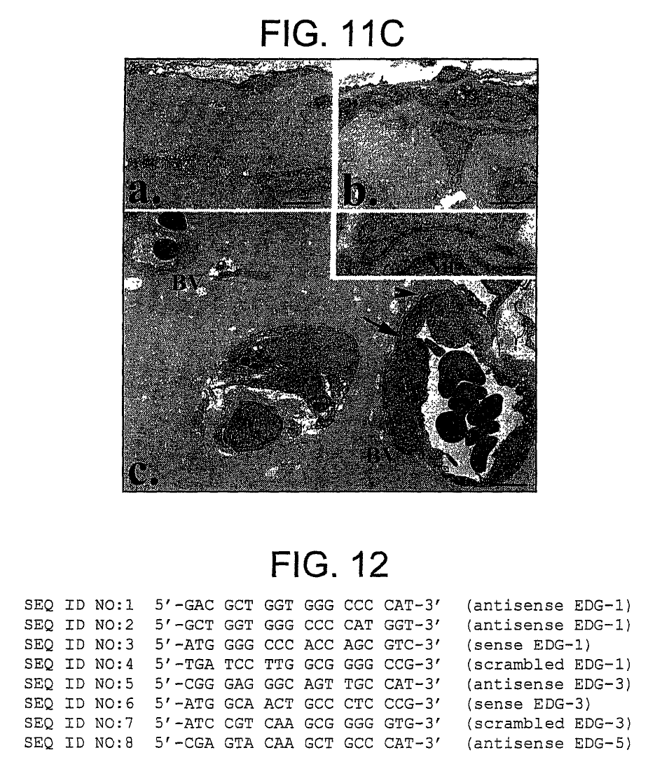

As shown in FIGS. 10A-C, SPP potentiates FGF-2-induced angiogenesis in vivo. Panels a and b of FIG. 11A show the low power micrograph of angiogenic response in implanted MATRIGEL plugs, whereas panels c-h show the histological analysis of sections of MATRIGEL plugs using hematoxylin-eosin staining. Panels a and d, FGF-2 alone; panels b, f and h, SPP+FGF-2; panel c, vehicle control; panel e, SPP alone; panel g, sphingosine (SPH)+FGF. Panel h is a high power view of the boxed area in panel f. SPP significantly enhanced the density and maturation of vascular vessels induced by FGF-2 (arrows). Arrowhead in (a) indicates the border of the plug. Scale bars in panels b, g, and h represent 320, 40, and 12.8 microns, respectively.

FIG. 11B shows quantification of neo vessels using the MATRIGEL plug in vivo assay. In these experiments, MATRIGEL plugs were fixed, dehydrated, embedded, and sections were subjected to hematoxylin and eosin staining. Angiogenesis was quantified by direct counting of vascular structures as described. Vascular density for each treatment was quantified and represented as mean±standard deviation (n=4). Data are from a representative experiment, which was repeated twice. As shown by the data in FIGS. 11A-B, SPP dramatically enhanced FGF-2 induced angiogenesis; and vascular density and the appearance of mature vascular structures were greatly increased by SPP.

Transmission electron microscope analysis indicated that neovessels with well-developed adherens junctions were increased by the FGF-2 and SPP treatment (FIG. 11C). The inset shows the higher magnification view of adherens junctional structure between to endothelial cells (arrow in panel c). Arrowheads denote the basement membrane of neovessels (Bv) induced by SPP, wherein Nu=nuclei. Scale bars are 5 microns in A, B, C, 0.5 microns in inset.

### Example 12

### Inhibition of Angiogenesis by Phosphothioate Oligonucleotide Treatment

A series of 18-mer phosphothioate oligonucleotides (PTO) were synthesized as potential antisense blocking agents to inhibit the expression of EDG-1 and EDG-3 receptors. The specificity and efficacy of the PTOs were tested in Xenopus oocytes programmed to express EDG-1 and EDG-3 receptors (Ancellin, N., and Hla, T., J. Biol. Chem. 274: 18997-19002 (1999)). Briefly, oocytes were injected with 20 nL of capped messenger RNA (EDG-1+G_{qi} chimeric G protein, 1 mg/mL of each; EDG-3, 50 ng/mL) premixed with the indicated PTO (100 ng/mL in water). Thirty-two hours after injection, oocytes were injected with photoprotein Aequorin (20 nL of 1 mg/mL) and stimulated with 20 nM of SPP. Light emission was recorded for 90 seconds with a luminometer (Turner design). Each experiment was repeated at least three times with multiple oocytes from different frogs.

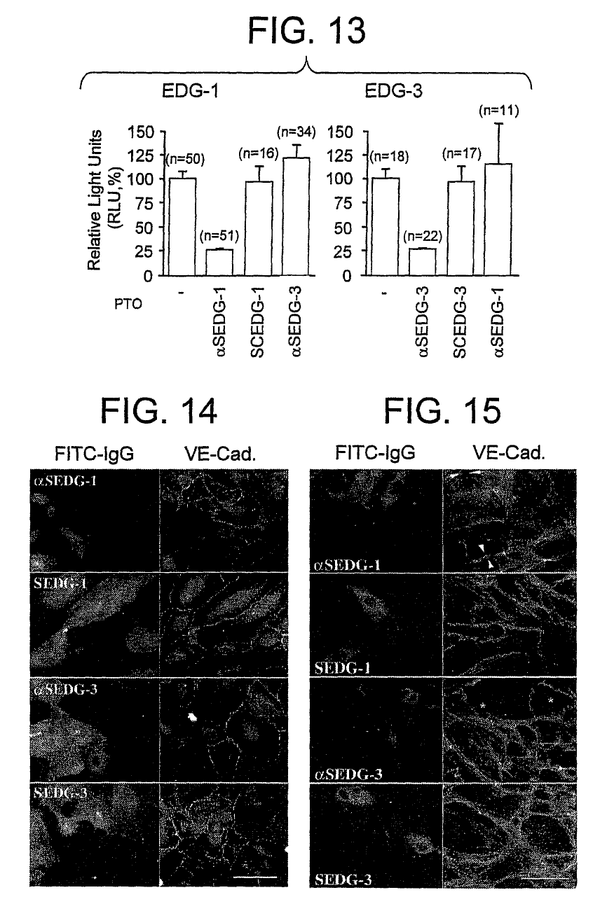

As shown in FIG. 13, coinjection of EDG-1 antisense PTO with the EDG-1 cRNA resulted in profound inhibition of EDG-1 expression as determined by suppression of SPP-induced calcium rises (Ancellin, N., and Hla, T., J. Biol. Chem. 274: 18997-19002 (1999)). EDG-3 antisense PTO did not inhibit the EDG-1 cRNA for EDG-3. These data suggest that the EDG-1 and EDG-3 PTOs are specific inhibitors of respective receptor expression.

To examine the effects on HUVEC, PTOs and FITC-IgG were microinjected into the cytoplasm of HUVEC cells using the Eppendorf Transjector microinjector system as described by Macrez-Lepretre et al., J. Biol. Chem., Vol. 272, 10095-10102 (1997). Alternatively, PTOs were delivered into HUVEC by Lipofectin reagent (Life Technologies, Inc.), essentially as described by Ackermann, E., et al., J. Biol. Chem. 274: 11245-11252 (1999).

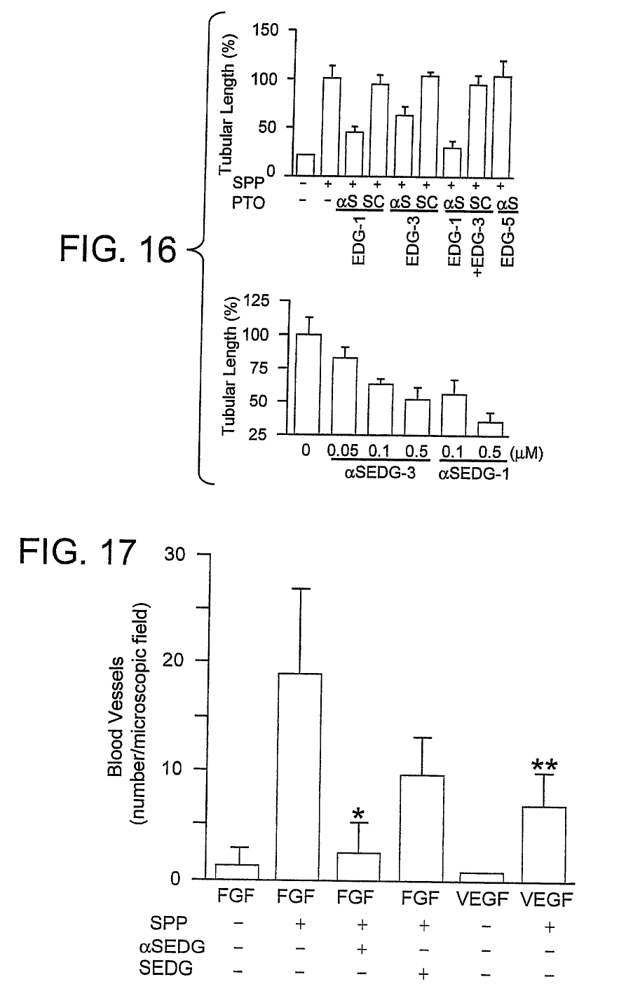

These reagents were microinjected into the cytoplasm of HUVEC to block the expression of EDG-1 and -3 receptors, and SPP-induced VE-cadherin assembly into cell-cell junctions was analyzed. As shown in Table 1 and FIG. 14, both EDG-1 and -3 antisense PTOs inhibited the SPP-induced VE-cadherin localization at cell-cell junctions. Co-administration of both EDG-1 and EDG-3 antisense PTOs attenuated SPP-induced HUVEC morphogenesis in an additive manner. In contrast, neither the complementary nor the scrambled sequence of EDG-1 and -3 oligonucleotides inhibited VE-cadherin assembly significantly. Administration of the EDG-5 antisense PTO also did failed to block the SPP-induced endothelial cell morphogenesis (FIG. 16).

TABLE 1

| | | Cortical Actin | Stress Fiber | VE-Cadherin |
|---|---|---|---|---|
| Treatments | Amount | (% of cells inhibited) | (% of cells inhibited) | (% of cells inhibited) |
| αS-EDG-1 (SEQ ID NO:1) | 40 μM | 79 ± 12 (n = 90)* | 36 ± 15 (n = 90) | 89 ± 9 (n = 120)* |
| αS-EDG-1 (SEQ ID NO:1) | 20 μM | 64 ± 7 (n = 90)* | 17 ± 8 (n = 90) | 72 ± 11 (n = 200)* |
| αS-EDG-1 (SEQ ID NO:1) | 10 μM | ND | 11 ± 10 (n = 40) | 67 ± 14 (n = 70)* |
| S-EDG-1 (SEQ ID NO:3) | 40 μM | 25 ± 7 (n = 70) | 21 ± 13 (n = 70) | 21 ± 11 (n = 90) |
| S-EDG-1 (SEQ ID NO:3) | 20 μM | ND | 13 ± 6 (n = 30) | 16 ± 13 (n = 70) |
| SC-EDG-1 (SEQ ID NO:4) | 20 μM | 13 ± 6 (n = 200) | 14 ± 5 (n = 200) | 9 ± 7 (n = 260) |
| αS-EDG-3 (SEQ ID NO:5) | 20 μM | 4 ± 2 (n = 40) | 66 ± 9 (n = 40)* | 53 ± 10 (n = 170)* |
| S-EDG-3 (SEQ ID NO:6) | 20 μM | 8 ± 6 (n = 140) | 12 ± 8 (n = 140) | 8 ± 5 (n = 150) |
| αS-EDG-5 (SEQ ID NO:8) | 20 μM | 12 ± 7 (n = 230) | 13 ± 8 (n = 230) | 10 ± 7 (n = 160) |
| FITC-IgG | | 12 ± 8 (n = 220) | 11 ± 9 (n = 210) | 9 ± 8 (n = 210) |

αS = antisense, S = sense, SC = scrambled.
(*) indicates statistical significance (p < 0.01, t test) of PTOs treatment versus control (FITC-IgG alone).
n = numbers of cells scored. ND, not determined.

Antisense EDG-1 PTO furthermore attenuated the formation of cortical actin structures in HUVEC, which are known to be induced by the Rac pathway (FIG. 15). In contrast, formation of stress fibers was specifically inhibited by antisense EDG-3 PTO. These data support the notion that induction of the Rac pathway by EDG-1 and the Rho pathway by EDG-3 are necessary for SPP-induced adherens junction assembly.

The presence of EDG-1 and EDG-3 PTOs will also inhibit SPP-induced morphogenesis, as shown in FIG. 16.

Finally, the effect of the presence of EDG-1 and EDG-3 PTOs and VEGF on SPP-induced angiogenesis is shown in FIG. 17.

### Example 13

### Akt Binds the i₃ Domain of EDG-1

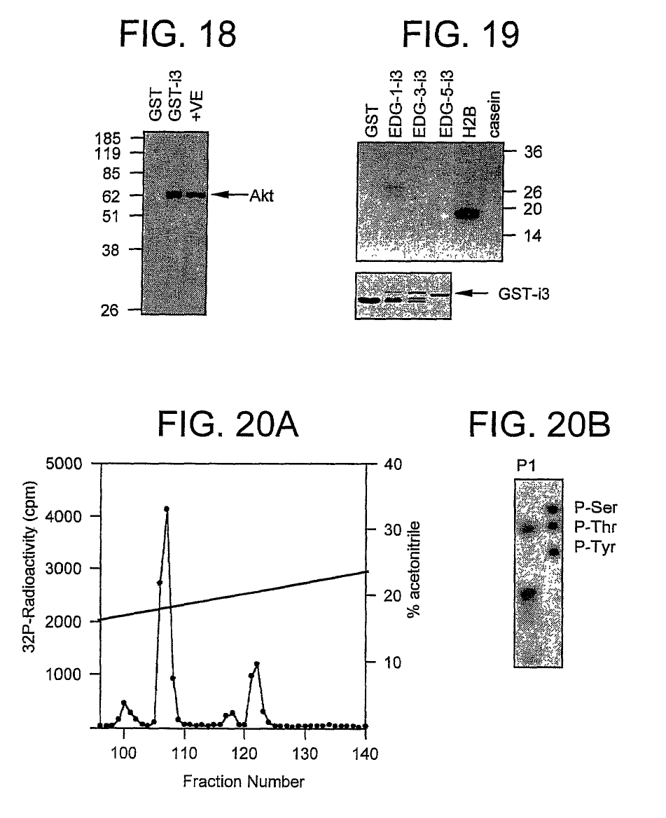

As shown in FIG. 18, glutathione S-transferase (GST)-EDG-1-i₃ but not GST associated with Akt. For the detection of Akt/EDG-1 association, cells were stimulated with or without ligand, and the protein complexes were covalently linked in situ by 0.5 mM DSP (dithiobis[succinimidyl propionate]; Pierce) for 15 min. Cellular extracts ware prepared and immunoprecipitated as described above. The immunoprecipitated complexes were released by incubating in sample buffer (20% β-mercaptoathanol) at room temperature for 1 hr and addition of 10 μl of 1 M DTT to the gel lanes before separation by SDS-PAGE.

### Example 14

### Akt Phosphorylates the T²³⁶ Residue of EDG-1

Whether Akt is capable of phosphorylating the i₃ domain of EDG-1 was next tested. The GST-i₃ polypeptides from EDG-1, -3, and -5 subtypes of S1P receptors were prepared and incubated with active Akt enzyme in vitro. As shown in FIG. 19, only the GST-EDG-1-i₃ was phosphorylated by Akt. For in vitro phosphorylation reactions, two micrograms of GST fusion polypeptides were incubated with 1 U/ml recombinant, active Akt (Alessi et al. FEBS Letters 399, 333-338, (1996)) in kinase buffer containing 50 mM Tris-HCl (pH 7.5), 10 mM magnesium acetate, 100 μM ATP (10 μCl [γ-³²P]ATP), 0.1 mM EGTA, 1 μM Microcystin-LR. After incubation at 30° C. for 15 min, reactions were stopped by adding 5× SDS-PAGE sample buffer and resolved on SDS-PAGE.

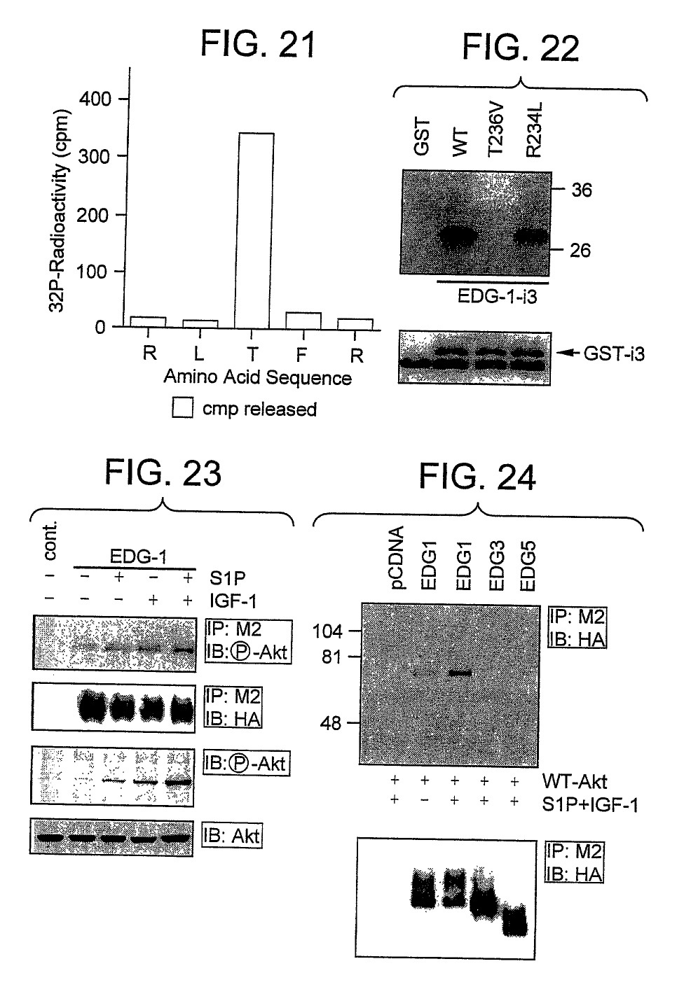

Digestion of the labeled GST-EDG1-i₃ with trypsin followed by chromatography on a C18 column revealed the presence of a major labeled tryptic phosphopeptide, termed P1, eluting at 18% acetonitrile (FIG. 20). Phosphoamino acid analysis revealed that P1 contained only phosphothreonine. After solid phase sequencing, ³²P radioactivity was released after the third cycle of Edman degradation (FIG. 21). The sequence of P1 was determined by gas-phase Edman sequencing of this peptide. The molecular mass of P1, determined by MALDI-TOF mass spectrometry (772.41), was identical to that expected for the tryptic phosphopeptide comprising residues 234-238, phosphorylated at T²³⁶. This site matched the known Akt consensus sequence (Alessi et al. FEBS Letters 399, 333-338, (1996)). A small peak, termed P2, eluting at about 20% acetonitrile only appeared when high concentrations of Akt were used in the phosphorylation reaction. Sequence analysis of P2 indicated that it is a phosphoserine-containing tryptic peptide corresponding to the sequence (RGSR²²³IYSL²²⁷). This is an artificial sequence created by fusion of the linker “RGS” sequence between the GST and the i₃ domain of EDG-1. It is distantly related to the Akt consensus sequence.

When the T²³⁶ site was mutated to V, ³²P-phosphate incorporation into the GST-i₃T236V polypeptide was significantly reduced upon incubation with Akt (FIG. 22), suggesting that T²³⁶ is a unique Akt phosphoacceptor site in the i₃ domain of EDG-1. These data indicate that Akt binds to the i₃ domain of EDG-1 and specifically phosphorylates the T²³⁶ residue in vitro.

In order to map the site on GST-EDGI-i₃ phosphorylated by Akt, 2 mg GST-EDG1-i₃ was incubated with 1 U/ml Akt in a reaction containing 50 mM Tris-HCl (pH 7.5), 0.1 mM EGTA, 0.1% (v/v) β-mercaptoethanol, 10 mM magnesium acetate, 100 μM [γ-³²P]ATP (10000 cpm/pmol), and 1 μM microcystin-LR. The reactions were terminated by adding 1% SDS, 10 mM dithiothreitol, and heated at 100° C. for 5 min. After cooling, 4-vinylpyridine was added to a concentration of 2% (by volume), and the sample was left on a shaking platform for 30 min at 30° C. to alkylate cysteine residues, The sample was subjected to electrophoresis on a 4%-2% NuPAGE Bis-Tris gel, and the 29 kDa ³²P-labeled band corresponding to GST-EDG1-1₃ was excised and cut into small pieces. These were washed sequentially for 15 min on a vibrating platform with 1 ml of the following: water, a 1:1 mixture of water and acetonitrile, 0.1 M ammionium bicarbonate, a 1:1 mixture of 0.2 M ammonium bicarbonate and acetonitrile, and finally acetonitrile. The gel pieces were dried by rotary evaporation and incubated in 0.3 ml of 50 mM ammonium bicarbonate, 0.05% (by mass) Zwittergent 3-16 containing 2 mg of alkylated trypsin. After 16 hr, the supernatant was removed and the gel pieces were washed for 10 min in a further 0.3 ml of 50 mM ammonium bicarbonate, 0.05% (by mass) Zwittergent 3-16. The supernatants were then combined, and after adding 0.1% (by volume) trifluoroacetic acid, chromatographed on a Vydac 218TP54 C18 column (Separations Group, Hesperia, Calif.).

The site of phosphorylation of Peptide P1 (20) was determined by solid-phase Edman degradation of the peptide coupled to Sequelon-AA membrane (Milligen) as described previously (Stokoe et al., EMBO J, 11: 3985-3994 (1992)). The sequence identity of this peptide was confirmed by Edman sequencing on Applied Biosystems 476A sequenator. Peptide P1 was also analyzed by MALDI-TOF mass spectrometry on a PerSeptive Biosystems Elite-STR mass spectrometer using α-cyanocinnamic acid as the matrix, Spectra were obtained in both the linear and reflector mode.

### Example 15

### Akt Binds and Phosphorylates Intact EDG-1 in an Activation-Dependent Manner

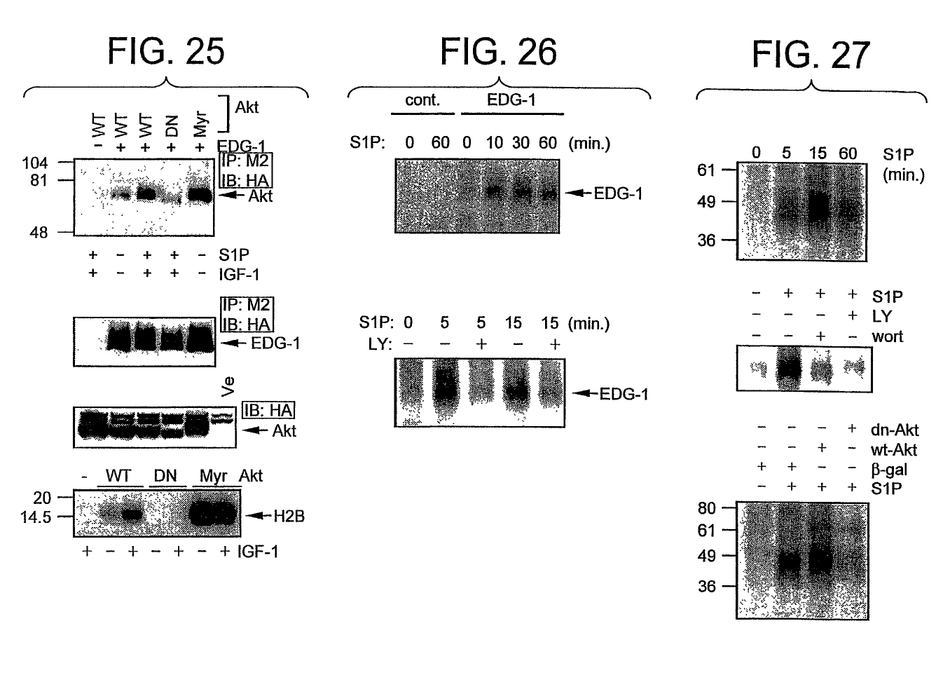

Whether SPP- or RTK-induced Akt activation would influence association between EDG-1 and Akt was tested. As shown in FIG. 23, SPP or IGF-1 (a P1K ligand which is known to be a strong activator of Akt) increased EDG-1-associated Akt. Both factors induced EDG-1 association of Akt in an additive manner. These data suggest that SPP or IGF-1 activation of endogenous Akt results in binding to EDG-1. Furthermore, only EDG-1 but not EDG-3 and -5 associated with Akt, and the association was significantly enhanced when Akt was activated by S1P and IGF-1 (FIG. 24). In agreement, dominant-negative Akt did not associate with EDG-1, whereas constituitively active Akt bound to EDG-1 even in the absence of SPP and IGF-1 (FIG. 25).

Next, whether SPP treatment induces EDG-1 phosphorylation was examined. In HEK293EDG-1 cells, S1P induced a time-dependent phosphorylation of EDG-1 (FIG. 26). Furthermore, the phosphorylation of EDG-1 was suppressed by the P1-3-Rinase inhibitor LY294002, consistent with the notion that Akt is activated by the P1-3-kinase pathway.

To label the EDG-1 receptor in HUVEC and HEK293EDG-1 cells, they were pre-incubated in phosphate-free DMEM (GIBCO-BRL) for 2 hr. Subsequently, cells were labeled with [³²P] orthophosphate (80 μCl/ml) for 3 hr. To examine the effect of P1-3 kinase, cultures were treated with wortmannin (100 nM) or LY294002 (10 μM) during tine last hour of labeling. After SPP stimulation, ³²P-labeled EDG-1 receptor was immunoprecipitated with either chicken anti-EDG-1 1gY or anti-M2, resolved on SDS-PAGE, and visualized by autoradiography.

### Example 16

### Akt Induces Phosphorylation of Endogenously Expressed EDG-1

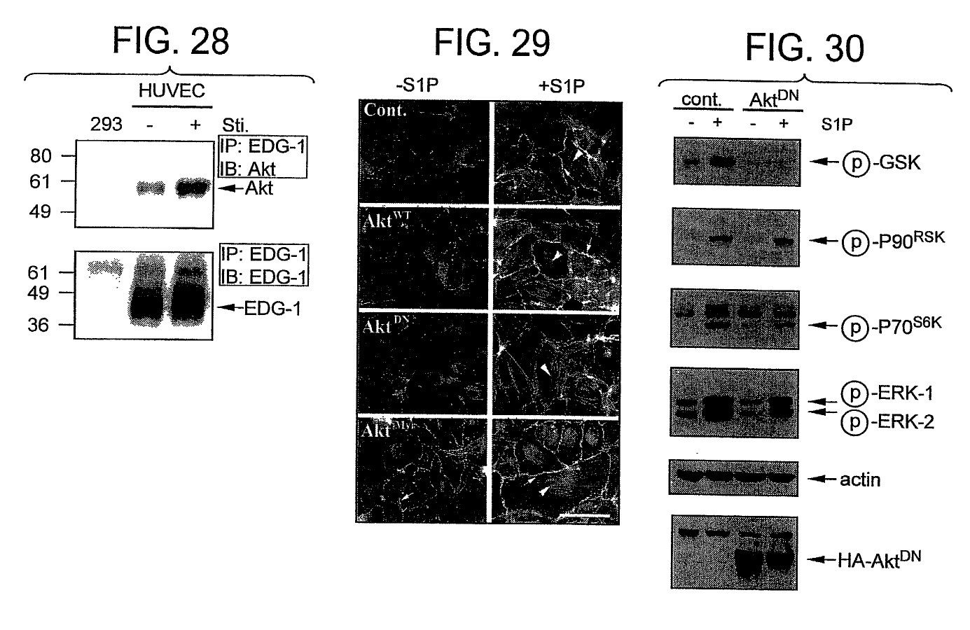

Chicken anti-human EDG-1 antibodies against human EDG-1 polypeptide expressed in E. coli were raised in Aves Labs, Tigard, Oreg. This antibody specifically detected EDG-1 polypeptide in HUVEC cells and transfected HEK293 cells. When ³²P-orthophosphate-labeled HUVEC cells were treated with S1P for 5-60 min and cell lysates were immunoprecipitated with the anti-EDG-1 antibody, radiolabeling of the EDG-1 polypeptide was increased (FIG. 27). This stimulation of EDG-1 phosphorylation was inhibited by wortmannin, LY294002, and transduction of the dominant-negative Akt virus. Furthermore, stimulation of HUVEC cells followed by immunoprecipitation of endogenously expressed EDG-1 results in the increased association with Akt (FIG. 28). Together, these data strongly suggest that activation of Akt results in its association and concomitant phosphorylation of EDG-1.

Chicken IgY against human EDG-1 was bound to protein-A beads by using the rabbit anti-IgY (Jackson lmmunoResearch Labs). After washing, the immunoglobulin-protein A beads were covalently linked by the DMP (dimethyl pimelimidate; Pierce) reagent. Cells were solubilized for 45 mm with extraction buffer (60 mM octyl glucopyranoside, 1% Triton X-100, 0.15 M NaCl, 10 mM Tris [pH 8.0], 10 mM MgCl₂) containing 1 μM Microcystin, 50 mM NaF, 10 mM β-glycerophosphate, 5 mM sodium pyrophosphate, 1 mM sodium orthovanadate, and protease inhibitor cocktail (Calbiochem). After centrifugation at 15,000× g for 15 min, 1 mg of cell extracts was immunoprecipitated with antibody beads. For the detection of Akt/EDG-1 association, cells were stimulated with or without ligand, and the protein complexes were covalently linked in situ by 0.5 mM DSP (dithiobis[succinimidyl propionate]; Pierce) for 15 min. Cellular extracts ware prepared and immunoprecipitated as described above. The immunoprecipitated complexes were released by incubating in sample buffer (20% β-mercaptoathanol) at room temperature for 1 hr and addition of 10 μl of 1 M DTT to the gel lanes before separation by SDS-PAGE.

### Example 17

### Requirement for Akt Activation in SPP/EDG-1-Induced Cortical Actin Assembly and Migration

To determine the effect of the Akt activity on SPP-induced responses in HUVEC, we utilized the adenoviral transduction approach using wild-type Akt, dominant-negative Akt, and constitutively active Akt (myr-Akt). As shown in FIG. 29, weak cortical actin structures were induced by wild-type Akt overexpression. Myr-Akt induced strong cortical actin structures in the absence of SPP, and dominant-negative Akt blocked SPP-induced cortical actin structures. The effect of dominant-negative Akt overexpression in HUVEC was specific since SPP-induced phosphorylation of GSK-3β was blocked, whereas phosphorylation of other AGC kinases, such as p90^{rsk}, p70^{86k}, and p42/44^{erk-1/-2} was not inhibited by the dominant-negative Akt overexpression (FIG. 30). These data suggest that Akt activation by S1P is important in cortical actin assembly in HUVEC.

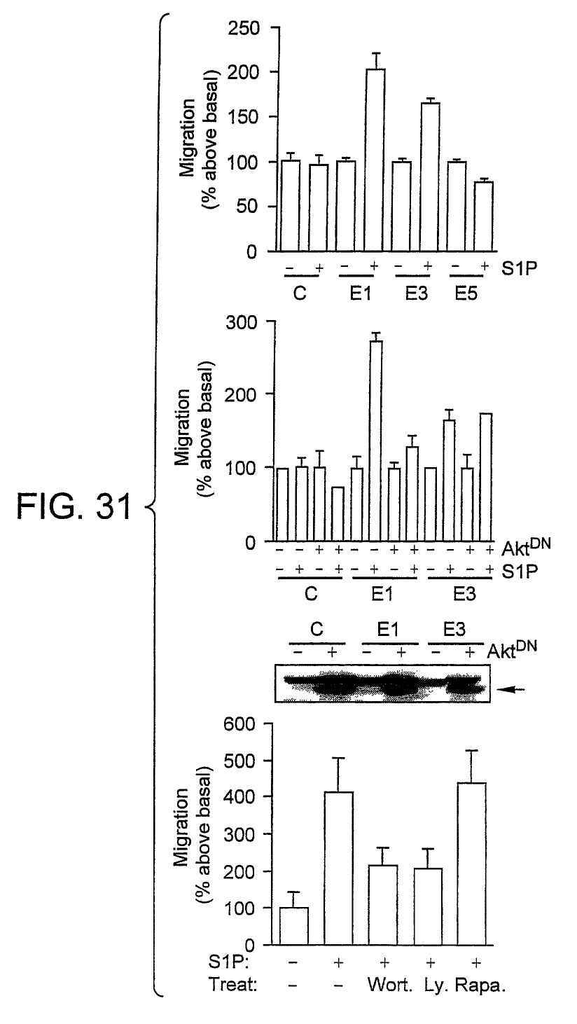

The role of Akt signaling in cell migration induced by SPP in CHO cells overexpressing the EDG-1 and -3 receptors was examined. CHO cells do not express the endogenous SPP receptors. However, S1P was able to induce CHO cell migration when EDG-1 and -3 but not EDG-5 were expressed (FIG. 31). The migration response in EDG-1-expressing cells was strongly inhibited by dominant-negative Akt, wortmannin, and LY294002. In contrast, the EDG-3 response was not affected. These observations indicate that the Akt is required for EDG-1-induced cortical actin assembly and cell migration.

CHO and HEK293 cells were transfected with LipofectAmine-2000 reagent (GIBCO-BRL) according to manufacturer's instructions. To establish the stably-transfected cultures, CHO cells transfected with pCDNAneo, EDG-1, or EDG-3 plasmids were selected in HAMS F-12 supplemented with 10% FBS, G418 (1 mg/ml; GIBCO-BRL). EDG-5 transfectant was selected with Zeocin (1 mg/ml; Invitrogen). Receptor expression was detected by immunoprecipitating 1 mg of cellular extracts with anti-Flag epitope antibody (M2) followed by Western blotting with the same antibody. Mutant receptors were prepared by standard oligonucleotide-mediated site-directed mutagenesis protocols (Promega Biotec) and confirmed by DNA sequencing. The construction of adenoviral transducing particles and procedure for 5,, transduction were essentially as described (Fulton et al. Nature 339: 597-601, (1999)).

### Example 18

### The Akt-Defective EDG-1 Mutant (T236AEDG-1) Activates the Gi Pathway and Associates with Akt

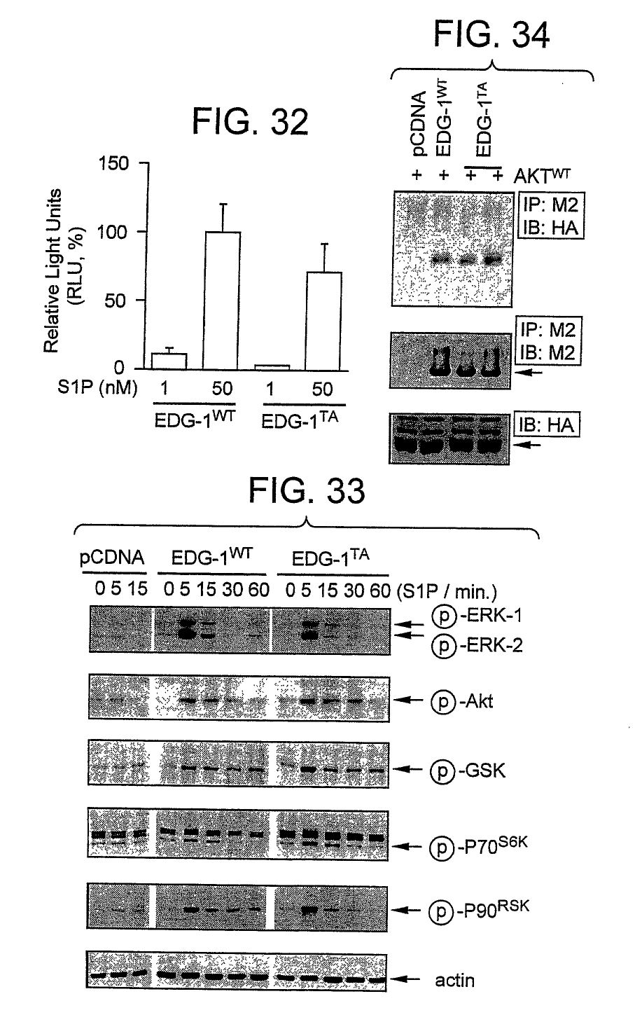

In order to define the function of the Akt phosphorylation of the T²³⁶ residue in the function of the EDG-1 receptor, we mutated the T236 residue to A by site-directed mutagenesis. Mutant receptors were prepared by standard oligonucleotide-mediated site-directed mutagenesis protocols (Promega Biotec) and confirmed by DNA sequencing. When mRNA encoding wild-type or T236AEDG-1 together with the heterotrimeric G₁ protein was expressed in Xenopus oocytes and the oocytes were stimulated with S1P, intracellular calcium rises were observed, suggesting that the T236A mutation does not impair coupling to the G₁ pathway (FIG. 32). When CHO cells expressing EDG-1 or T236AEDG-1 were stimulated with S1P, phosphorylation of G₁-dependent kinases, such as p42/44^{ERK-1/-2}, Akt, GSK-3β, p70^{86K}, and p90^{RSK}, was observed to a similar extent and kinetics (FIG. 33). Moreover, the T236AEG-1 mutant receptor associated with the Akt, similar to the wild-type EDG-1 (FIG. 34). These data suggest that the Akt phosphorylation-defective EDG-1 mutant still associates with Akt and is capable of signaling rapid cellular responses, such as intracellular calcium rises and G₁/P1-3-kinase dependent phosphorylation events.

### Example 19

### Akt Phosphorylation-Defective EDG-1 Mutant Fails to Activate Rac, Cortical Actin Assembly and Cell Migration in Response to SPP

The protein kinase Akt exhibits a strict substrate specificity, in that the -3 and -5 residues relative to the phosphoacceptor site must be arginine (R) residues. In contrast, related kinases, such as p70^{86K} and p90^{RSK}, will phosphorylate substrates in which the -3 and -5 residues are lysine (K). To obtain further evidence of the requirement for Akt phosphorylation in EDG-1 signaling, the T236AEDG-1 mutant and the Akt consensus site mutants (R231K and R233K) were prepared. All three mutants were phosphorylated very poorly by the active Akt kinase in an in vitro phosphorylation reaction when compared to the wild-type EDG-1 (FIG. 35). In contrast, in vitro phosphorylation with p90^{RSK} was similar in wild-type as well as in the three mutants, suggesting that this kinase phosphorylates EDG-1 in vitro at a site(s) distinct from the T236 residue.

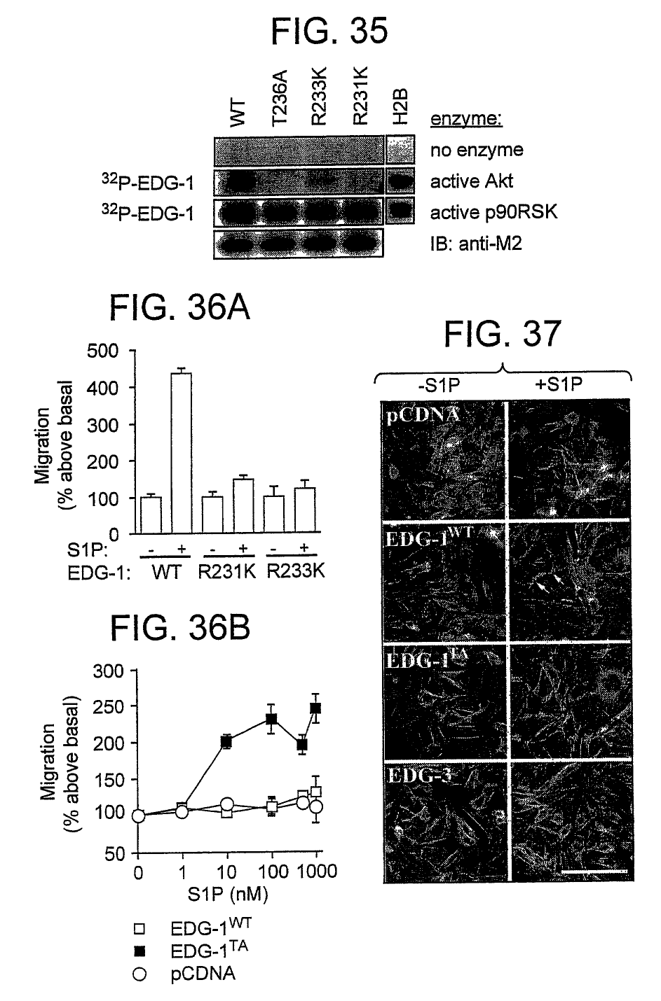

To test the role of these mutants in EDG-1 signaling, CHO stable cells lines expressing these mutant receptors were derived. As shown in FIG. 36A, SPP induced cell migration in wild-type EDG-1-transfected CHO cells but not in cells transfected with T236A, R231K, and R233K mutants. Dose-response studies (36 B) showed that a wide range of S1P concentrations (1 nM to 1 μM) failed to induce CHO cell migration in the T236A mutant.

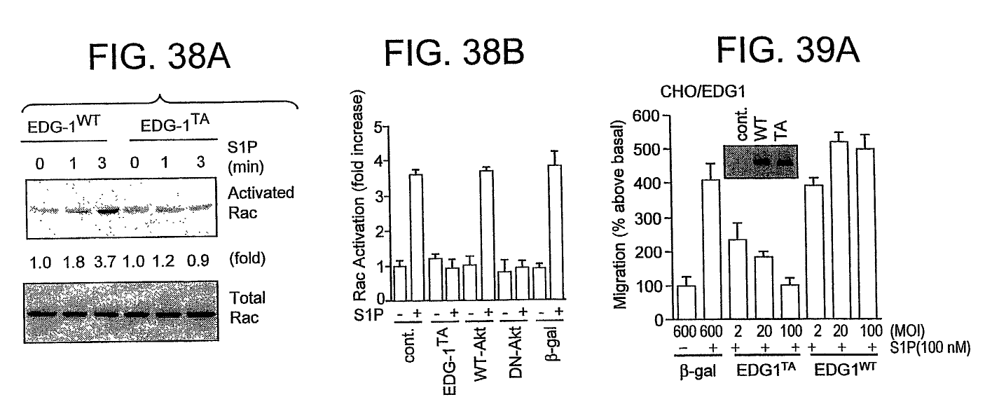

The molecular basis of the migration defect in Akt phosphorylation site mutant EDG-1 receptor was next examined. SPP was unable to induce cortical actin structures in T236AEDG-1-transfected CHO cells (FIG. 37), suggesting a defect in this pathway. Both wild-type and mutant transfected cells attached to the substratum and spread normally upon SPP treatment (FIG. 37), suggesting that a specific defect in EDG-1 signaling is involved. Furthermore, the T236AEDG-1 receptor was not able to activate the Rac GTPase (FIG. 38). Indeed, SPP-induced Rac activation in the EDG-1-expressing cells was sensitive to inhibition of Akt activity (FIG. 38). Moreover, the T236AEDG-1 receptor over expression blocked S1P-induced Rac activation (FIG. 38). These data suggest that EDG-1 needs to be phosphorylated on the T236 residue by the protein kinase Akt to induce specific downstream signaling pathways important for Rac activation, cortical actin assembly and chemotaxis.

Rac activation and cell migration assays were performed as described in Paik et al. J. Biol. Chem. 276: 11830-11837, (2001)). To visualize actin microfilaments; cells were fixed with 4% formaldehyde, permeabilized with 0.2% Triton-X 100, and stained with TRITC-phalloidin (0.06 μg/ml; Sigma).

### Example 20

### Akt Phosphorylation Mutant EDG-1 (T236AEDG-1) Acts as a Dominant-Negative G-Protein Coupled Receptor

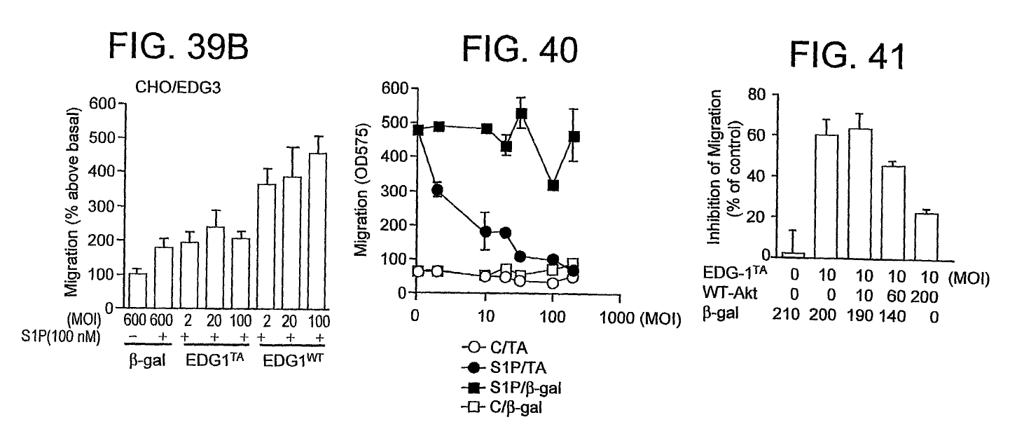

This receptor was tested to see if it would act functionally as a dominant-negative GPCR by sequestering the Akt. As shown in FIG. 39A, T236AEDG-1 but not the wild-type EDG-1 virus inhibited EDG-1-dependent chemotaxis in response to S1P. In contrast, SPP-induced chemotaxis in EDG-3-expressing CHO cells was not affected, consistent with the knowledge that Akt does not bind or phosphorylate this receptor (FIG. 39B). Furthermore, transduction of wild-type EDG-1 potentiated the SPP induced migration in EDG-3-expressing CHO cells. The effect of the T236AEDG-1 virus on HUVEC cell responses to SPP was also tested. The T236AEDG-1 virus but not the β-gal virus inhibited S1P-induced 1-HUVEC migration (FIG. 40).

To further substantiate the notion that the T236AEDG-1 mutant receptor acts as a dominant-negative GPCR by sequestering Akt, we tested the effect of increasing levels of Akt in endothelial cells to overcome the suppressive effects of the T236AEDG-1 receptor. As shown in FIG. 41, Akt expression, in a dose-dependent manner, restored up to about 65% of chemotaxis inhibition in HUVEC cells. These data suggest that the T236AEDG-1 receptor is an effective dominant-negative GPCR and that it suppresses endothelial cell migration by sequestering Akt and thereby uncouples the receptor to activate the Rac GTPase.

### Example 21

### Akt Phosphorylation Mutant EDG-1 (T236AEDG-1) Acts as a Dominant-Negative G-Protein Coupled Receptor and Inhibits Angiogenesis

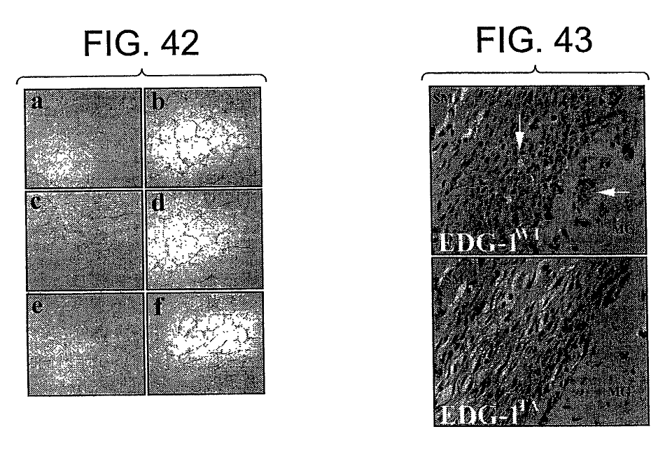

Endothelial cell migration is an essential component of angiogenesis. Since the T236AEDG-1 mutant inhibited endothelial cell migration, we tested if this construct will block angiogenesis in vitro and in vivo. As shown in FIG. 42, the T236AEDG-1 mutant but not the wild-type EDG-1 receptor inhibited morphogenesis of HUVEC cells plated on Matrigel. In addition, the T236AEDG-1 mutant but not the wild-type EDG-1 receptor inhibited the FGF-2- and SPP-induced angiogenesis in the Matrigel model of in vivo angiogenesis in nude mice. Histological analysis (FIG. 43) indicates that invasion/migration of the neovessels into Matrigel plugs is significantly inhibited by the T236AEDG-1 virus. These data strongly suggest that Akt-mediated phosphorylation of EDG-1 is critical for endothelial cell migration and angiogenesis in vivo.

All references cited herein are incorporated by reference, as well as Lee, Menq-Jer, et al., Cell, Vol. 99, 301-312 (1999), and Lee, Meng-Jer et al Molecular Cell, Vol. 8, 1-20 (2001).

It should be understood that the foregoing relates only to preferred embodiments of the present invention and that numerous modifications or alterations may be made therein without departing from the spirit and scope of the invention For example, structural analogues of SPP are likely to have similar activity to SPP itself.

What is claimed is:

1. A method to inhibit angiogenesis in vivo, comprising administration of a composition comprising a pharmaceutically effective quantity of an antagonist of EDG-1 signal transduction.

2. The method of claim 1, wherein the composition further comprises at least one additional anti-angiogenic factor.

3. The method of claim 1, wherein the composition further comprises a PI-3-kinase inhibitor.

4. The method of claim 1, wherein the composition further comprises an Akt kinase inhibitor.

5. The method of claim 1, wherein the composition further comprises wortmannin.

6. The method of claim 1, wherein the composition further comprises LY294002.

7. The method of claim 1, wherein the composition further comprises the DNA sequence encoding a mutated EDG-1 receptor.

8. The method of claim 7, wherein the mutated EDG-1 receptor is T236A, R231K or R233K.

9. A method for treatment of unwanted angiogenesis in a human or animal, comprising administration of a composition comprising a pharmaceutically effective quantity of an antagonist of EDG-1 signal transduction.

10. The method of claim 9, wherein the composition further comprises an anti-EDG-1 antibody.

11. The method of claim 10, wherein the anti-EDG-1 antibody is a chicken-anti-human-EDG-1 antibody.

12. The method of claim 10, wherein the anti-EDG-1 antibody is a biologically active fragment.
